(19) United States
(12) Patent Application Publication
(10) Pub. No.: US 2023/0105741 A1
(43) Pub. Date: Apr. 6, 2023

(54) PHOTOREACTIVE AND CLEAVABLE PROBES FOR TAGGING BIOMOLECULES

(71) Applicant: Syncell (Taiwan) Inc., Taipei City (TW)

(72) Inventors: Hsiao-Jen CHANG, New Taipei City (TW); Yi-De CHEN, Yuanshan Township (TW); Chih-Wei CHANG, New Taipei City (TW); Chantal Hoi Yin CHEUNG, Taipei City (TW); Yu-Chih LIN, Taipei City (TW); Chia-Wen CHUNG, Taipei City (TW); Hsiang-Ju KAI, Taipei City (TW)

(21) Appl. No.: 17/933,460

(22) Filed: Sep. 19, 2022

Related U.S. Application Data
(60) Provisional application No. 63/246,283, filed on Sep. 20, 2021.

(51) Int. Cl. G01N 33/53; G01N 33/68

(52) U.S. Cl. CPC G01N 33/53; G01N 33/68; G01N 2333/948

(57) ABSTRACT

Compositions including photoreactive and cleavable probes and methods of using the probes. The probes may include a tag conjugatable to a label, a cleavable linker linkable to a bait molecule, and a light-activated warhead. The compositions and methods may be useful for analyzing biomolecules.

### PRIORITY CLAIM

This patent application claims priority to U.S. provisional patent application No. 63/246,283, titled “PHOTOREACTIVE AND CLEAVABLE PROBES FOR TAGGING BIOMOLECULES” and filed on Sep. 20, 2021, which is herein incorporated by reference in its entirety.

### SEQUENCE LISTING

The instant application contains a Sequence Listing which has been submitted electronically in XML file format and is hereby incorporated by reference in its entirety. Said XML copy, created on Dec. 9, 2022, is named 14815-701_200_SL.xml and is 57,431 bytes in size.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD

Described herein are methods and compositions for identifying, tagging, and analyzing biomolecules. Specifically described are cleavable probes useful for photoactivated and tagging of subsets of biomolecules. The methods and compositions may be particularly useful for analyzing biological samples, such as identifying proximal biomolecules in cell or tissue samples.

### BACKGROUND

Cells are composed of different types of biological molecules (biomolecules). The biomolecules in the cells interact with neighbor biomolecules in the subcellular environment to form complexes, organelles, or other assemblies and to carry out various essential cell functions. Characterizing the subcellular environment, within which biomolecules interact with one another, and how the biomolecules function together is very challenging. Biomolecules are small, and they exist in a cell environment with tens of millions of other molecules. The interactions between neighboring biomolecules are frequently weak, and techniques used to study biomolecules disrupt their interactions. While techniques such as yeast two-hybridization assays and more recently proximity labeling have advanced our understanding of the cell environment, these techniques suffer from various limitations such as nonspecific binding, slow reaction times and disruption of the natural cell environment, resulting in false positives and missed interactions. What is needed are better tools for determining naturally occurring biomolecule interactions. Described herein are systems, compositions, and methods to better analyze endogenous biomolecule interactions.

### SUMMARY OF THE DISCLOSURE

Described herein are systems, compositions, and methods to better analyze endogenous biomolecule interactions. The methods and compositions may be useful for identifying, tagging, and analyzing biomolecules. Specifically described are cleavable probes useful for photoactivated and tagging of subsets of biomolecules. The methods and compositions may be particularly useful for analyzing biological samples, such as identifying proximal biomolecules in cell or tissue samples. These probes may be especially useful for selectively tagging and proximity labeling of biomolecules via selective light illumination through a microscope system. In general, in one embodiment, a photoreactive and cleavable probe of formula (I)

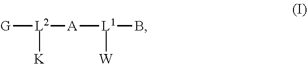

wherein a cleavable linker of the probe includes an L¹ portion at a proximal region of the cleavable linker, an L² portion at a distal region of the cleavable linker, an A portion including a cleavage site, W includes a light-activated warhead covalently bound to the proximal region of the cleavable linker, B includes a tag bound to the proximal region of the cleavable linker, K includes a crosslinkable group bound to the distal region of the cleavable linker, and G includes a bait molecule bound to the distal region of the cleavable linker. This and other embodiments can include one or more of the following features. The bait molecule can include an antibody and the antibody can be bound to the distal region of the cleavable linker. The bait molecule can include a secondary antibody and the secondary antibody can be bound to the distal region of the cleavable linker. The bait molecule can include a CLIP-tag, a HaloTag, protein A, protein G, protein L, an RNA molecule, a small molecule, or a SNAP-tag. The crosslinkable group can include a click chemistry-based moiety. The crosslinkable group can include a bioorthogonal moiety. The crosslinkable group can include a strained alkyne, a terminal alkyne, an azide, a tetrazine, a strained alkene, or a 2-cyano-6-aminobenzothiazole (CBT) moiety. The cleavable linker can include a cleavable linker bond other than a disulfide bond. The cleavable linker can include an azobenzene derivative, a boronic acid ester, a Dde derivative, a DNA oligomer, or a specifically cleavable peptide. The cleavable linker can include the azobenzene derivative, and the azobenzene derivative can include the moiety of

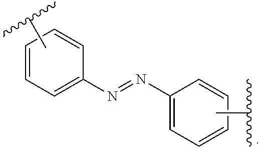

The cleavable linker can include the Dde derivative, and the Dde derivative can include the moiety of

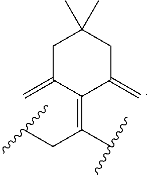 [SMILES: *CC(*)=C1C(=C)CC(C)(C)CC1=C]

The cleavable linker includes a specifically cleavable peptide. The cleavable linker can include a bioorthogonal protease-cleavable peptide chain. The cleavable linker can include a human rhinovirus 3C (HRV 3C) protease recognition sequence or a tobacco etch virus (TEV) protease recognition sequence. The tag can include a biotin derivative, a click chemistry tag, a CLIP-tag, a digoxigenin tag, a HaloTag, a peptide tag, or a SNAP-tag. The tag can include a click chemistry tag, and the click chemistry tag includes an alkyne-based or azide-based moiety. The tag can include a click chemistry tag, and the click chemistry tag can include the moiety of

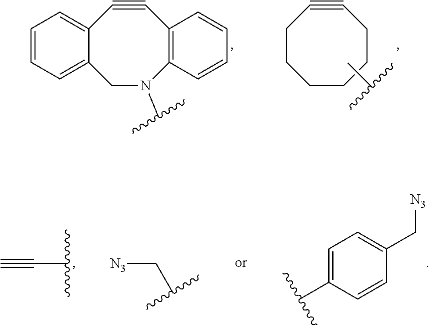

The tag can include a biotin derivative, and the biotin derivative includes the moiety of

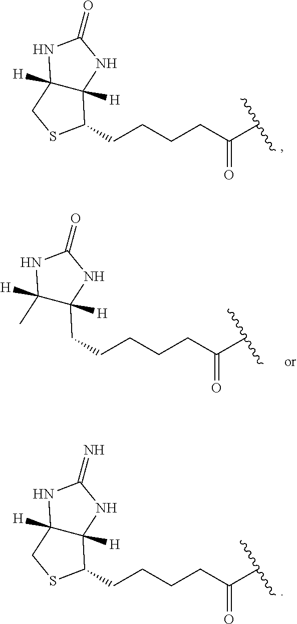

The light-activated warhead can include an aryl azide, a benzophenone, or a diazirine. The light-activated warhead can include the aryl azide, and the aryl azide includes the moiety of

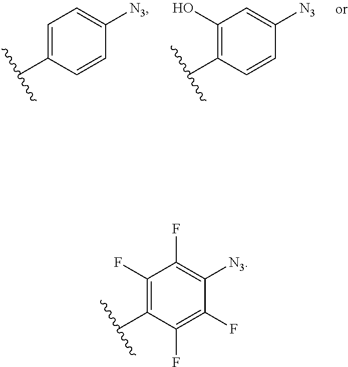 [SMILES: *c1ccc(N=[N+]=[N-])cc1 ; *c1ccc(N=[N+]=[N-])cc1O ; *c1c(F)c(F)c(N=[N+]=[N-])c(F)c1F]

The light-activated warhead can include the benzophenone, and the benzophenone includes the moiety of

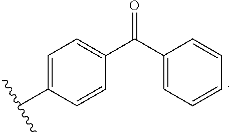 [SMILES: *c1ccc(C(=O)c2ccccc2)cc1]

The light-activated warhead includes the diazirine, and the diazirine includes the moiety of

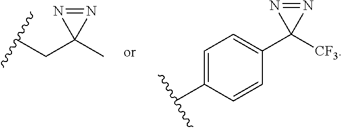

The light-activated warhead includes a nucleobase-specific 3-cyanovinylcarbazole nucleoside (CNVK), and the CNVK includes the moiety of

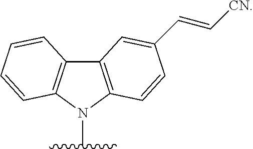 [SMILES: *n1c2ccccc2c2cc(/C=C/C#N)ccc21]

The light-activated warhead includes a nucleobase-specific psoralen, and the psoralen includes the moiety of

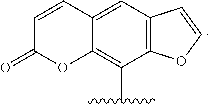 [SMILES: *c1c2occc2cc2ccc(=O)oc12]

The light-activated warhead can include a phenoxyl radical trapper, and the phenoxyl radical trapper includes the moiety of

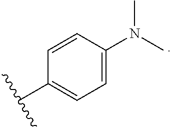 [SMILES: *c1ccc(N(C)C)cc1]

The L1 portion can include a moiety of formula (I-1), and B and W are bound to the proximal region of the cleavable linker through the moiety of formula (I-1)

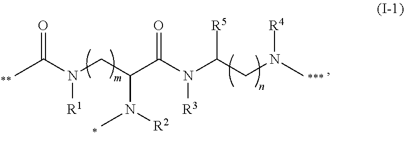

wherein m and n each independently are 1, 2, 3, 4, 5, or 6, the symbol * is a first attachment site for a proximal end of the “A” portion, the symbol ** is a second attachment site for one of either the tag or the light-activated warhead, the symbol *** is a third attachment site for the other one of the tag or the light-activated warhead, R¹, R², R³ and R⁴ each independently are hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, or a nitrogen protecting group, and R⁵ is —COORa, wherein Ra is hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, or an oxygen protecting group. The L² portion can include a moiety of formula (I-2), and the G and the K are bound to the distal region of the cleavable linker through the moiety of formula (I-2):

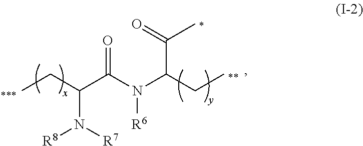

wherein x and y each independently are 1, 2, 3, 4, 5, or 6, the symbol * is a first attachment site for a distal end of the “A” portion, the symbol ** is a second attachment site for one of the crosslinkable group and the bait molecule, the symbol *** is a third attachment site for another one of the crosslinkable group and the bait molecule, and R⁶, le and R⁸ each independently are hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, or a nitrogen protecting group. The probe can include the following structure (SEQ ID NO: 37):

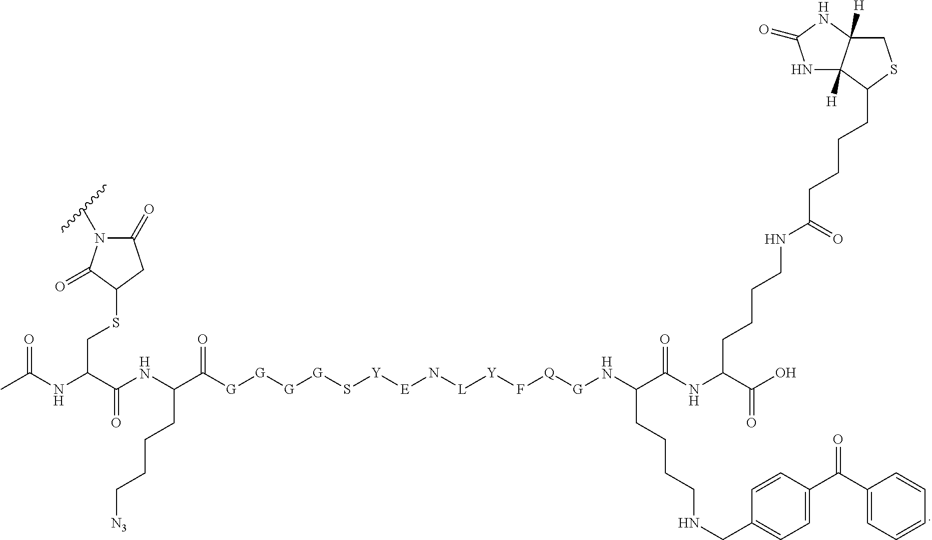

In general, in one embodiment, a connector molecule includes a warhead binding region wherein the warhead binding region preferentially binds to a light-activated warhead, and a crosslinkable moiety, wherein the crosslinkable moiety preferentially binds to a crosslinkable moiety binding partner.

This and other embodiments can include one or more of the following features. The warhead binding region can preferentially bind an aryl azide light-activated warhead, a benzophenone light-activated warhead, or a diazirine light-activated warhead. The warhead binding region can include an aliphatic C—H region. The crosslinkable moiety can include a click chemistry-based moiety. The crosslinkable moiety can include a bioorthogonal moiety. The crosslinkable moiety can include a strained alkyne, a terminal alkyne, an azide, a tetrazine, a strained alkene, or a 2-cyano-6-aminobenzothiazole (CBT) moiety.

In general, in one embodiment, kit for labeling biomolecules including a photoreactive and cleavable probe as above in a first container, and/or a connector molecule as above in a second container, and an instructional material.

In general, in one embodiment, a method for photoactivated labeling, the method including delivering a photoreactive and cleavable probe as above to a sample, conjugating the bait molecule to a target biomolecule in the sample, delivering a connector molecule to the sample, wherein the connector molecule includes a warhead binding region and a crosslinkable moiety, selectively illuminating a selected region of interest of the sample with optical radiation, to thereby activate the light-activated warhead and attaching, in the selected region of interest of the sample, the light-activated warhead to the warhead binding region, crosslinking the crosslinkable group on the probe with the crosslinkable moiety on the connector molecule to thereby crosslink the tag to the bait through the crosslink, cleaving the cleavable linker of the probe with a cleaver, such that tag crosslinked to the bait through the crosslink remains attached to the bait, while tag that is not crosslinked to the bait is removed from the bait, and removing unbound and cleaved probe and connector molecules.

In general, in one embodiment, an analytical method includes delivering a photoreactive and cleavable probe to a biological sample, wherein the probe includes a bait molecule, a cleavable linker, a light-activated warhead, a tag, and a crosslinkable group, wherein the light-activated warhead and the tag are attached to a proximal region of the cleavable linker, and the crosslinkable group and bait are attached to a distal region of the cleavable linker, conjugating the probe to a target biomolecule in the biological sample to form a conjugated probe-target biomolecule, illuminating the biological sample from an imaging lighting source of an image-guided microscope system, imaging the illuminated sample with a controllable camera, acquiring with the camera at least one image of subcellular morphology of the biological sample in a first field of view, processing the at least one image and determining a region of interest in the sample based on the processed image, obtaining coordinate information of the region of interest, selectively illuminating the region of interest with optical radiation to activate the light-activated warhead and attach the warhead to a warhead binding region of a connector molecule, wherein the connector molecule further includes a crosslinkable moiety, crosslinking the crosslinkable group on the probe with the crosslinkable moiety on the connector molecule to thereby crosslink the tag to the bait, cleaving the cleavable linker of the probe at a cleavage site between the proximal and distal regions of the cleavable linker, such that tag crosslinked to the bait through the crosslink remains attached to the bait, while tag that is not crosslinked to the bait is removed from the bait, and removing unbound and cleaved probe and connector molecules.

This and other embodiments can include one or more of the following features. The step of cleaving the cleavable linker can include performing a bioorthogonal cleavage reaction. The cleavable linker can include a cleavable linker bond and the step of cleaving the cleavable linker can include cleaving a bond other than a disulfide bond. The step of conjugating a detectable label with the tag of the probe and detectably proximity labeling neighbors proximal the target biomolecule by detectable label activity. The step of selectively illuminating can include illuminating a region for 25 us/pixel to 400 us/pixel, for 50 us/pixel to 300 us/pixel, or for 75 us/pixel to 200 us/pixel. The step of selectively illuminating can include illuminating with a power intensity of from 100 mW to 300 mW. The step of selectively illuminating can include illuminating a zone defined by point spread function. The step of detectably proximity labeling can include photoselective proximity labeling a region less than 300 nm, less than 200 nm, or less than 100 nm in diameter. The detectable label can include a catalytic label. The biological sample can include at least one, at least 100, at least 1000 or at least 10,000 live or fixed cells. The biological sample can include fixed cells, fixed tissues, cell extracts, or tissue extracts. The biological sample can be disposed on a microscope stage, the method can further include removing at least a portion of the biological sample region of interest from the stage. The method further can further include subjecting the sample to mass spectrometry analysis or sequencing analysis. The tag can include a biotin derivative, a CLIP-tag, a click chemistry tag, digoxigenin tag, a HaloTag, a peptide tag, or a SNAP-tag. The cleavable linker can include an azobenzene derivative, a boronic acid ester, a Dde derivative, a DNA oligomer, or a peptide. The bait molecule can include an antibody, a CLIP-tag, a HaloTag, protein A, protein G, protein L, a small molecule, or a SNAP-tag. light-activated warhead includes an aryl azide, a benzophenone, or a diazirine.

The photoreactive and cleavable probe of formula (I),

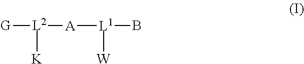

wherein a cleavable linker of the probe includes an L′ portion at a proximal region of the cleavable linker, an L² portion at a distal region of the cleavable linker, an A portion comprising a cleavage site, W includes a light-activated warhead covalently bound to the proximal region of the cleavable linker, B includes a tag bound to the proximal region of the cleavable linker, K includes a crosslinkable group bound to the distal region of the cleavable linker, and G includes a bait molecule attachment region bound to the distal region of the cleavable linker, wherein the bait molecule attachment region selectively binds to a bait molecule. The bait molecule attachment region can include an antibody attachment region. The bait molecule can include a CLIP-tag, a HaloTag, protein A, protein G, protein L, an RNA molecule, a small molecule, or a SNAP-tag. The crosslinkable group can include a click chemistry-based moiety. The crosslinkable group can include a bioorthogonal moiety. The crosslinkable group can include a strained alkyne, a terminal alkyne, an azide, a tetrazine, a strained alkene, or a 2-cyano-6-aminobenzothiazole (CBT) moiety. The cleavable linker can include a cleavable linker bond other than a disulfide bond. The cleavable linker can include an azobenzene derivative, a boronic acid ester, a Dde derivative, a DNA oligomer, or a specifically cleavable peptide. The cleavable linker can include the azobenzene derivative, and the azobenzene derivative includes the moiety of

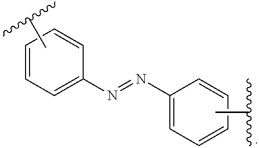

The cleavable linker can include the Dde derivative, and the Dde derivative includes the moiety of

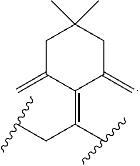

The cleavable linker can include a specifically cleavable peptide. The cleavable linker can include a bioorthogonal protease-cleavable peptide chain. The cleavable linker can include a human rhinovirus 3C (HRV 3C) protease recognition sequence or a tobacco etch virus (TEV) protease recognition sequence. The tag can include a biotin derivative, a click chemistry tag, a CLIP-tag, a digoxigenin tag, a HaloTag, a peptide tag, or a SNAP-tag. The tag can include a click chemistry tag, and the click chemistry tag includes an alkyne-based or azide-based moiety. The tag can include a click chemistry tag, and the click chemistry tag includes the moiety of

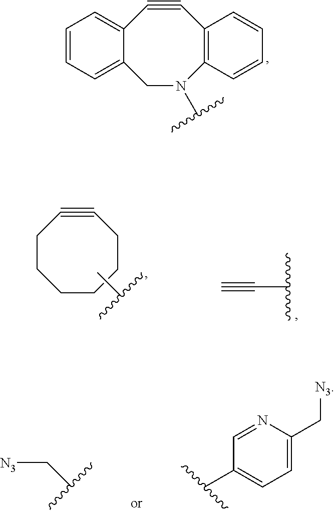

The tag can include a biotin derivative, and the biotin derivative can include the moiety of

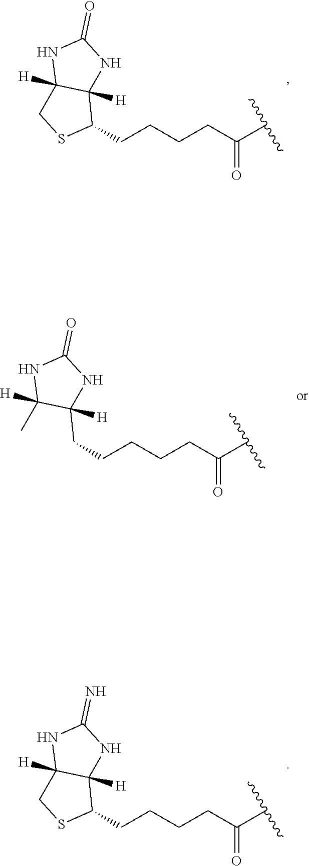

The light-activated warhead can include an aryl azide, a benzophenone, or a diazirine. The light-activated warhead can include the aryl azide, and the aryl azide includes the moiety of

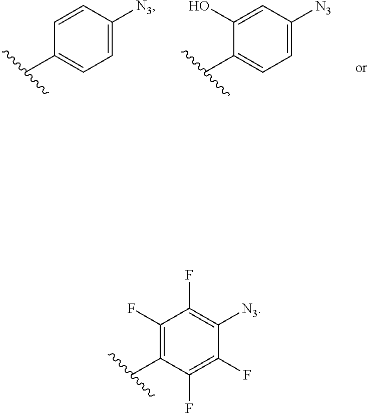 [SMILES: *c1ccc(N=[N+]=[N-])cc1 ; *c1ccc(N=[N+]=[N-])cc1O ; *c1c(F)c(F)c(N=[N+]=[N-])c(F)c1F]

The light-activated warhead includes the benzophenone, and the benzophenone includes the moiety of

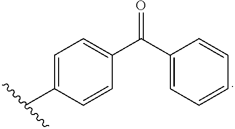 [SMILES: *c1ccc(C(=O)c2ccccc2)cc1]

The light-activated warhead includes the diazirine, and the diazirine includes the moiety of

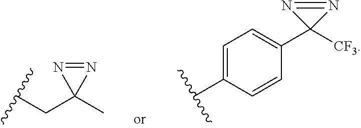

The light-activated warhead includes a nucleobase-specific 3-cyanovinylcarbazole nucleoside (CNVK), and the CNVK includes the moiety of

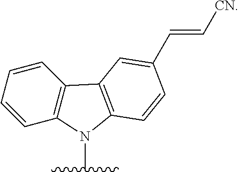 [SMILES: *n1c2ccccc2c2cc(/C=C/C#N)ccc21]

The light-activated warhead includes a nucleobase-specific psoralen, and the psoralen includes the moiety of

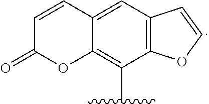 [SMILES: *c1c2occc2cc2ccc(=O)oc12]

The light-activated warhead includes a phenoxyl radical trapper, and the phenoxyl radical trapper includes the moiety of

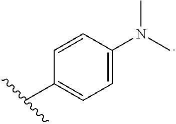 [SMILES: *c1ccc(N(C)C)cc1]

The L1 portion can include a moiety of formula (I-1), and B and W are bound to the proximal region of the cleavable linker through the moiety of formula (I-1):

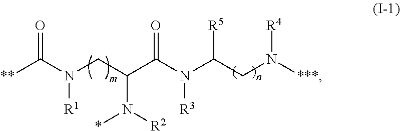

wherein m and n each independently are 1, 2, 3, 4, 5, or 6, the symbol * is a first attachment site for a proximal end of the “A” portion, the symbol ** is a second attachment site for one of the tag and the light-activated warhead, the symbol *** is a third attachment site for another one of the tag and the light-activated warhead, R¹, R², R³ and R⁴ each independently are hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, or a nitrogen protecting group, and R⁵ is —COORa, wherein Ra is hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, or an oxygen protecting group. The L² portion includes a moiety of formula (I-2), and the G and the K are bound to the distal region of the cleavable linker through the moiety of formula (I-2)

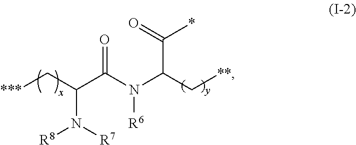

wherein x and y each independently are 1, 2, 3, 4, 5, or 6, the symbol * is a first attachment site for a distal end of the “A” portion, the symbol ** is a second attachment site for one of the crosslinkable group and the bait molecule, the symbol *** is a third attachment site for another one of the crosslinkable group and the bait molecule, and R⁶, R⁷ and R⁸ each independently are hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, or a nitrogen protecting group. The photoreactive and cleavable probe can include the following structure (SEQ ID NO: 37):

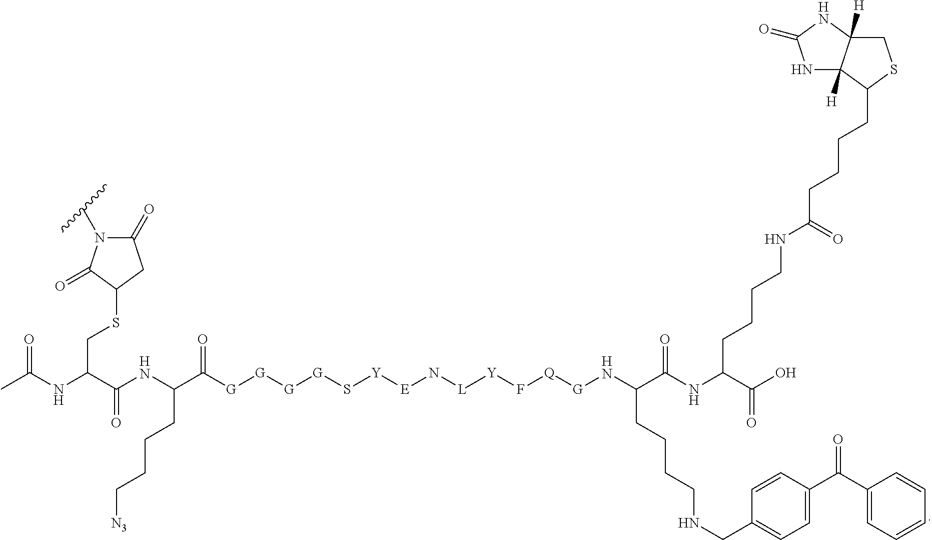

In general, in one embodiment, kit for labeling biomolecules includes the photoreactive and cleavable probe as above in a first container and/or the connector molecule as above in a second container, and an instructional material.

### BRIEF DESCRIPTION OF THE DRAWINGS

A better understanding of the features and advantages of the methods and apparatuses described herein will be obtained by reference to the following detailed description that sets forth illustrative embodiments, and the accompanying drawings of which:

FIG. 1 shows a schematic depiction of a system useful for photoselective spatial tagging and proximity labeling of cells on a substrate.

FIG. 2A shows a schematic illustration of a multifunctional photoreactive and cleavable probe. The photoreactive and cleavable probe has a multivalent core with a plurality of attachment sites. A tag, a cleavable linker, and a light-activated warhead are bound to the attachment sites on the probe. FIG. 2B schematically illustrates a proximity labeling system that can be used to label biomolecules in a small region of interest using the probe shown in FIG. 2A.

FIG. 2C shows a schematic illustration comparing the results of direct photochemical labeling with photo-assisted enzymatic proximity labeling using the multifunctional photoreactive and cleavable probes described herein to label biomolecules in small region of interest (ROI). The probes are shown in FIG. 2B.

FIG. 3A and FIG. 3B schematically illustrate the effects on protein structure using a multifunctional photoreactive and cleavable probe and mild cleavage conditions as described herein (FIG. 3B), compared with results using a probe with harsh cleavage reactions (FIG. 3A). With mild cleavage conditions the protein structure is retained, and the reaction is bioorthogonal, while with harsh cleavage conditions, the protein denatures, and the reaction is non-bioorthogonal.

FIGS. 4A-4K show examples of tags that can be used in the photoreactive and cleavable probes described herein. The tags are configured to interact with a label for labeling biomolecules neighboring a target molecule of interest. FIG. 4A-FIG. 4E show examples of click chemistry tags that can be used with the probes. FIGS. 4F-4H show examples of biotin derivatives that can be used with the probes. FIG. 4I shows a digoxigenin moiety. FIG. 4J shows a peptide tag (SEQ ID NO: 1). FIG. 4K shows a SNAP-tag.

FIGS. 5A-5E show examples of site-specific cleavable linkers that can be used in the photoreactive and cleavable probes described herein. FIG. 5A shows an azobenzene moiety. FIG. 5B shows a boronic ester moiety. FIG. 5C shows a Dde moiety. FIG. 5D shows a DNA oligomer. FIG. 5E shows a peptide moiety.

FIGS. 6A-6E shows examples of bait molecules that can be used in the photoreactive and cleavable probes described herein to conjugate with a molecule of interest in a sample. FIG. 6A shows an antibody that can be used a bait molecule. FIG. 6B shows a nucleic acid portion that can be used as a bait molecule. FIG. 6C shows a representation of a functional protein that can be used as a bait molecule. FIG. 6D shows small molecules/drugs can be used as bait molecules. By way of example, erlotinib is shown. FIG. 6E shows a CLIP-tag and other members of self-labeling moieties could be used (e.g., HaloTag or SNAP-Tag).

FIGS. 7A-7I show examples of photoactive warheads that can be used in the photoreactive and cleavable probes described herein.

FIG. 8A-8G show additional examples of linkers that can be used in the photoreactive and cleavable probe described herein.

FIGS. 9A-9G show examples of photoreactive and cleavable probes. The probes have multivalent cores with a plurality of attachment sites. A tag is bound to one of attachment sites, a cleavable linker is bound to another attachment site, and a light-activated warhead is bound to another of the attachment sites on the probe.

FIGS. 10A-10B schematically illustrate peptide-based photoreactive and cleavable probes. These probes have a peptide region cleavable by a peptide cleavage reagent, such as by a protease that recognizes a specific peptide sequence. FIG. 10A shows an example of a peptide-based probe with a tag and warhead on the N-terminal end of the peptide region. FIG. 10B shows an example of a peptide-based probe with a tag and warhead on the C-terminal end of the peptide region. FIGS. 10A-10B also show probes with an additional, flexible linker and an optional clickable amino acid. Additional linkers (also referred to as spacers) can play a role in bridging the attachment sites between bait molecules and photoreactive and cleavable probe. The distance between the probe and bait can be controlled by applying linkers with different spatial lengths.

FIGS. 10C-10I show examples of reactive or clickable amino acids that can be used with the probes shown in FIGS. 10A and 10B. A clickable amino acid may be useful for attaching a bait molecule, such as an antibody.

FIGS. 10J-10Q show examples of peptide-based photoreactive and cleavable probes schematically illustrated in FIGS. 10A-10B. The cleavage sites for the human rhinovirus 3C (HRV 3C) protease, tobacco etch virus (TEV) protease, and thrombin are shown with arrows. Figure discloses SEQ ID NOS 28-35, respectively, in order of appearance.

FIGS. 11A-11D illustrate methods and steps used to synthesize the photoreactive and cleavable probes described herein. The methods create probes with a tag, a cleavable linker, and a light-activated warhead. Figure discloses SEQ ID NOS 30 and 36, respectively, in order of appearance.

FIG. 12A schematically illustrates a photoreactive and cleavable probe conjugated to an antibody bait.

FIG. 12B and FIG. 12C schematically illustrates a reaction scheme for performing photoselective tagging of a molecule using a photoreactive and cleavable probe conjugated to an antibody bait for tagging proteins in the cell nucleolus. FIG. 12B illustrates how the reaction proceeds using controlled light. FIG. 12B illustrates how the cleavable probes are cleaved to reduce background in non-lighted areas.

FIG. 12D shows results from using the reaction schemes shown in FIG. 12A and FIG. 12B. The nucleolin protein is specifically tagged in the presence of light (top and right panels) but is not tagged in the absence of light (bottom panel).

FIG. 13A represents a schematic diagram of an imaging-guided system.

FIG. 13B depicts the optical path of the image-guided system of FIG. 13A.

FIG. 14A represents a schematic diagram of another imaging-guided system.

FIG. 14B depicts the optical path of the image-guided system of FIG. 14A.

FIG. 15A represents a schematic diagram of yet another imaging-guided system.

FIG. 15B depicts the optical path of the image-guided system of FIG. 15A.

FIGS. 16A-16D show reagents and processes useful for tagging, obtaining, and analyzing biomolecules and their neighboring biomolecules. FIG. 16A schematically illustrates a multifunctional probe useful for photo-induced capturing of a connector molecule. FIG. 16B schematically illustrates a connector molecule that can be used with the multifunctional probe illustrated in FIG. 16A. FIG. 16C schematically illustrates a multifunctional probe and connector during use. The light-activated warhead has been light-activated and bound to the connector molecule. A crosslinkable group on the probe has reacted (clicked) with a corresponding crosslinkable group on the connector molecule and the cleavable linker has been cleaved. FIG. 16D schematically shows how a multifunctional probe and probe can tag a biomolecule in the presence of activating light (top). In the absence of light, the biomolecule is not tagged.

FIG. 17A shows an example of a multifunctional probe, such as the one schematically illustrated in FIG. 16A. Figure discloses SEQ ID NO: 37.

FIG. 17B shows an example of a connector molecule, such as the one schematically illustrated in FIG. 16B. Figure discloses SEQ ID NO: 38.

FIG. 17C shows an example of a multifunctional probe and connector, such as the one schematically illustrated in FIG. 16C, joined and cleaved at the cleavable linker. Figure discloses SEQ ID NOS 39-40, respectively, in order of appearance.

### DETAILED DESCRIPTION

Described herein are systems, compositions, and methods useful for identifying, tagging, obtaining, and analyzing biomolecules and their neighboring biomolecules. The compositions and methods may be particularly useful for analyzing biomolecule interactions in biological samples, such as analyzing proteins, nucleic acids, carbohydrates, or lipids in cell or tissue samples. The compositions and methods utilize photoreactive and cleavable probes (e.g., bioorthogonally or mildly cleavable or enzyme-specific cleavage) that can label biomolecules and their neighboring biomolecules, while largely maintaining naturally occurring molecular structure in the biomolecules. The photoreactive and mildly cleavable probes described herein may be particularly useful for specifically labeling subsets of biomolecules in subcellular regions of cells using an image guided microscope with precision illumination control such as the system described in U.S. Patent Publication No. 2018/0367717, to enable automatic labeling of cellular biomolecules of interest. The probes can be used for in situ tagging of biomolecules such as proteins inside cells or tissues and that can be followed by tag transfer or proximity labeling such as using Tyramide Signal Amplification (TSA). The biomolecules can be further analyzed by analytical techniques such as mass spectrometry and sequencing. These probes may be especially useful for performing omics studies, such as genomics, proteomics, and transcriptomics, and for finding relevant biomarkers for diagnosis and treatment.

### Abbreviations and Definitions

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. “Amino acid analogs” refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Amino acids described herein may be conservatively substituted so long as conservatively substituted peptide enables the desired function (such as recognition by a protease). Examples of conservative substitutions include Thr, Gly, or Asn for Ser and His, Lys, Glu, Gln for Arg. Conservative substitutions are described in e.g., Molecular Cloning: A Laboratory Manual, Fourth Edition, Green and Sambrook, eds., Cold Spring Harbor Laboratory Press, Cold Spring Harbor 2014, as well as corrections and updates thereto.)

The term “antibody” refers to immunoglobulin and related molecules and includes monoclonal antibodies, polyclonal antibodies, monomers, dimers, multimers, multi specific antibodies (e.g., bispecific antibodies), heavy chain only antibodies, three chain antibodies, single chain Fv, nanobodies, etc., and also includes antibody fragments. An antibody may be a polyclonal or monoclonal or recombinant antibody. Antibodies may be murine, human, humanized, chimeric, or derived from other species. As used herein, when an antibody or other entity “specifically recognizes” or “specifically binds” an antigen or epitope, it preferentially recognizes the antigen in a complex mixture of proteins and/or macromolecules and binds the antigen or epitope with affinity, which is substantially higher than to other entities not displaying the antigen or epitope.

The term “aryl” refers to an aromatic ring system having a single ring (e.g., a phenyl group or a substituted phenyl group). Aryl groups of interest include, but are not limited to, groups derived from aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene and the like. In certain embodiments, an aryl group includes from 6 to 20 carbon atoms. In certain embodiments, an aryl group includes from 6 to 12 carbon atoms. Examples of an aryl group are phenyl and naphthyl.

The term “bait molecule” refers to a molecule that specifically interacts with a molecule of interest, which may be referred to as a target (or prey). Examples of bait molecules include an antibody, CLIP-tag, a drug, a nucleic acid, a fluorescent in situ hybridization (FISH) probe, protein A, protein G, protein L, protein A/G, protein A/G/L, another small molecule, and a SNAP-tag.

The term “binding” refers to a first moiety physically interacting with a second moiety, wherein the first and second moieties are in physical contact with one another.

The term “bioorthogonal” refers to not interfering with or not interacting with biology (e.g., being inert to biomolecules).

The term “bioorthogonal reaction” or “bioorthogonal cleavage reaction” refers to a reaction that proceeds under physiologically relevant conditions and compatibility with naturally occurring functional groups and typically with fast kinetics, tolerance to an aqueous environment, and high selectively. A bioorthogonal reaction proceeds under conditions configured to maintain naturally occurring molecular structure, such as protein folding or three-dimensional structure. A bioorthogonal reaction does not break cross-links between different regions of polypeptide chains in endogenous or sample proteins or peptides. For example, a bioorthogonal reaction does not break covalent bonds in naturally occurring functional groups (e.g., disulfide (—S—S— bonds) in cysteine side chains). A bioorthogonal cleavage linker or a cleavage linker in a bioorthogonal cleavage probe is configured for bioorthogonal cleavage, such as being compatible with using an enzyme or bond-specific chemicals configured to proceed bioorthogonally without breaking covalent bonds in naturally occurring functional groups.

The term “biotin derivative” refers to a biotin moiety, including biotin and variations of biotin, such as biotin with an open ring or substitutions. Typically, a biotin derivative is easily detectable with a biotin-binding entity or protein, such as avidin, NeutrAvidin, or streptavidin.

The term “catalyzed reporter deposition” (CARD) refers an enzyme catalyzed deposition of a detectable molecule on or near target biomolecules (e.g., carbohydrates, lipids, nucleic acids, or proteins). In some embodiments, the enzyme in an enzyme catalyzed deposition is horseradish peroxidase (HRP) and the detectable molecule is tyramide or digoxygenin (DIG).

The term “cleavable linker bond” refers to the chemical bond in a cleavable linker configured to be specifically cleaved by a cleavage reagent. Typically, a cleavable linker bond refers to a single bond; however, in some variations, a cleavable linker bond can refer to more than one bond, such as in the case of a double-stranded DNA cleavable linker cleavable by an endonuclease in which two strands of DNA are cleaved.

The term “click chemistry” refers to a chemical approach that easily joins molecular building blocks. Typically, click chemistry reactions are efficient, high-yielding, reliable, create few or no byproducts, and are compatible with an aqueous environment or without an added solvent. An example of click chemistry is cycloaddition, such as the copper(I)-catalyzed [3+2]-Huisgen 1,3-dipolar cycloaddition of an alkyne and azide leading to the formation of 1,2,3-triazole or Diels-Adler reaction. Click chemistry also includes copper free reactions, such as a variant using substituted cyclooctyne (see e.g., J. M. Baskin et al., Proc. Natl. Acad. Sci. U.S.A. 2007 Oct. 23, 104 (43), 16793-16797.) Other examples of click chemistry are nucleophilic substitutions; additions to C—C multiple bonds (e.g., Michael addition, epoxidation, dihydroxylation, aziridination); and nonaldol like chemistry (e.g., N-hydroxysuccinimide active ester couplings). Click chemistry reactions can be bioorthogonal reactions, but do not need to be.

The term “conjugate” refers to a process by which two or more molecules specifically interact. In some embodiments, a tag and a label conjugate. In some embodiments, a bait and a cleavable probe conjugate.

The term “conjugatable” refers to a molecule that can specifically come together with another molecule to which it can be conjugated. In some embodiments, a bait is conjugatable to a biomolecule of interest. In some embodiments a cleavable probe is conjugatable to a label.

The term “detectable label” refers to a compound or composition which is or is configured to be conjugated directly or indirectly to a molecule. The label itself may be detectable and be a directly detectable label (such as, e.g., fluorescent labels such as fluorescent chemical adducts, radioisotope labels, etc.), or the label can be indirectly detectable (such as, e.g., in the case of an enzymatic detectable label, the enzyme may catalyze a chemical alteration of a substrate compound or composition and the product of the reaction is detectable). Examples of detectable labels include e.g., a biotin label, a fluorescent label, horseradish peroxidase, an immunologically detectable label (e.g., a hemagglutinin (HA) tag, a poly-histidine tag), another light emitting label, and a radioactive label. An example of an indirect label is biotin, which can be detected using a streptavidin detection method.

The term “enzymatic cleavage reaction” refers to cleavage or hydrolysis of bonds in molecules mediated by an enzyme. Typically, enzyme mediated reactions cleave covalent bonds and lead to the formation of smaller molecules.

The term “immunoglobulin-binding protein” refers to immunoglobulin-binding bacterial proteins and variations of immunoglobulin-binding bacterial proteins. Examples include protein A, protein G, protein L, protein A/G, and protein A/G/L. Protein A and protein G and are bacterial proteins originally obtained from Staphylococcus aureus and Group G Streptococci, respectively, and have high affinity for the Fc region of IgG type antibodies. Protein A/G combines the binding domains of protein A and protein G. Protein A/G/L combines binding domains of protein A, protein G, and protein L. Immunoglobulin-binding proteins bind to specific domain of antibodies.

The term “instructional material” includes a publication, a recording, a diagram, a link, or any other medium of expression which can be used to communicate the usefulness of one or more compositions of the invention for its designated use. The instructional material of a kit of the invention may, for example, be affixed to a container which contains the composition or components or be shipped together with a container which contains the composition or components. Alternatively, the instructional material may be shipped separately from a container with the intention that the instructional material and a composition or component be used cooperatively by the recipient.

The term “label” refers to a molecule which produces or can be induced to produce a detectable signal. In some embodiments, a label produces a signal for detecting a neighboring biomolecule. Examples of labels that can be used include avidin labels, NeutrAvidin labels, streptavidin labels to detect a biotin tag.

The term “linker” refers to a structure which connects two or more substructures. A linker has at least one uninterrupted chain of atoms extending between the substructures. The atoms of a linker are connected by chemical bonds, typically covalent bonds.

The term “light-activated warhead” refers to a group with a moiety activatable by application of optical radiation. Examples of light-activated warheads include aryl azides, benzophenone, and diazirines. Once activated, a light-activated warhead can bind to a specific binding partner.

The terms “bound to”, “conjugated to”, “attached to” and “linked to” refer to being directly or indirectly bound/conjugated/attached/linked. For instance, a cleavable linker, a light-activated warhead and a tag can be directly bound to an attachment sites of a multivalent core or linker without intervening atoms, groups or moieties therebetween; alternatively, they may be indirectly bound to the attachment sites of the multivalent core or linker by one or more intervening atoms, groups or moieties therebetween. An intervening atom(s), group(s) or moieties may include, for example, one or more non-carbon atoms, groups, or moieties, or an unsubstituted or substituted alkylene or alkenylene group, which may include amine, amide, ether, ester or thioester linkages, and optionally be interrupted by one or more heteroatoms and/or rings, including aromatic rings optionally substituted. A light-activated warhead or tag can be directly bound to a cleavable linker without intervening atoms, groups, or moieties therebetween; alternatively, they may be indirectly bound to the cleavable linker by one or more intervening atoms, groups or moieties therebetween.

The term “mass spectrometer” refers to an instrument for measuring the mass-to-charge ratio of one or more molecules in a sample. A mass spectrometer typically includes an ion source and a mass analyzer. Examples of mass spectrometers includes matrix assisted laser desorption ionization (MALDI), continuous or pulsed electrospray (ES) ionization, ionspray, magnetic sector, thermospray, time-of-flight, and massive cluster impact mass spectrometry.

The term “mass spectrometry” refers to the use of a mass spectrometer to detect gas phase ions.

The term “mass spectrometry analysis” includes linear time-of-flight (TOF), reflectron time-of-flight, single quadruple, multiple quadruple, single magnetic sector, multiple magnetic sector, Fourier transform, ion cyclotron resonance (ICR) or ion trap.

The term “photoactivated” or “light-activated” refers to excitation of atoms by means of radiant energy (e.g., by a specific wavelength or wavelength range of light, UV light, etc.). In some examples, a photoactivated molecule forms a covalent linkage with another molecule or another part of itself within its immediate vicinity.

The term “peptide” refers to a polymer in which the monomers are amino acids and the monomers are joined together through amide bonds. A peptide is typically at least 2, least 5, least 10, least 20, least 50, least 100, or at least 500 or more amino acids long.

The term “photoreactive group” refers to a functional moiety, which, upon exposure to light (e.g., a specific wavelength or wavelength range of light, UV light, etc.) becomes activated. A photoreactive group typically forms a covalent linkage with a molecule within its immediate vicinity.

The term “proximity molecule” or neighboring molecule refers to a molecule that is near another molecule. A proximity molecule or neighbor molecule may bound to the molecule (e.g., covalently or non-covalently) or may be close by and not bound to the molecule.

The term “prey” refers to a binding partner of a bait molecule. For example, if an antibody is a bait, a corresponding protein to which the bait molecule can bind is the corresponding prey. In some embodiments, a bait can bind with a single prey. In some embodiments, a bait can bind with more than one prey.

The term “protein tag” refers to peptide sequences of amino acids. Protein tags can typically be conjugated to a label. An example of a protein tag is a “self-labeling” tag. Examples of self-labeling tags include BL-Tag, CLIP-tag, covalent TMP tag, HALO-tag, and SNAP-tag. SNAP-tag is a ˜20 kDa variant of the DNA repair protein O6-alkylguanine-DNA alkyltransferase that specifically recognizes and rapidly reacts with benzylguanine (BG) derivatives. During a labeling reaction, the benzyl moiety is covalently attached to the SNAP-tag, releasing guanine. CLIP-tag is a variation of SNAP-tag configured to react specifically with O2-benzylcytosine (BC) derivatives rather than benzylguanine (BG).

The term “secondary antibody” refers to an antibody that specifically recognizes a region of another antibody. A secondary antibody generally recognizes the F_{c} region of a particular isotype of antibody. A secondary antibody may also recognize the F_{c} from one or more particular species.

The term “small molecule” refers to low molecular weight molecules that include carbohydrates, drugs, enzyme inhibitors, lipids, metabolites, monosaccharides, natural products, nucleic acids, peptides, peptidomimetics, second messengers, small organic molecules, and xenobiotics. Typically, small molecules are less than about 1000 molecular weight or less than about 500 molecular weight.

The term “tag” refers to a functional group, compound, molecule, substituent, or the like, that can enable detection of a target molecule. A tag can enable a detectable biological or physiochemical signal that allows detection via any means, e.g., absorbance, chemiluminescence, colorimetry, fluorescence, luminescence, magnetic resonance, phosphorescence, radioactivity. The detectable signal provided due to the tag can be directly detectable due to a biochemical or physiochemical property of the tag moiety (e.g., a fluorophore tag) or indirectly due to the tag interaction with another compound or agent. Typically, a tag is a small functional group or small organic compound. In some embodiments, the employed tag has a molecular weight of less than about 1,000 Da, 750 Da, 500 Da or even smaller.

The term “tagging” refers to the process of adding a tag to a functional group, compound, molecule, substituent, or the like. Typically, tagging enables detection of a target molecule.

The term “tyramide signal amplification” (TSA), refers to a catalyzed reporter deposition (CARD) an enzyme-mediated detection method that utilizes catalytic activity of an enzyme (e.g., horseradish peroxidase) to catalyze inactive tyramide to highly active tyramide. The amplification can take place in the presence of low concentrations of hydrogen peroxide (H₂O₂). In some examples, tyramide can be labeled with a detectable label, such as fluorophore (such as biotin or 2,4-dinitrophenol (DNP)).

The practice of the techniques described herein may employ, unless otherwise indicated, conventional techniques and descriptions of chemistry, biochemistry, cell biology, immunology, molecular biology (including cell culture, recombinant techniques, sequencing techniques) and organic chemistry technology which are explained in the literature in the field (e.g., Molecular Cloning: A Laboratory Manual, Fourth Edition, Green and Sambrook, eds., Cold Spring Harbor Laboratory Press, Cold Spring Harbor 2014, as well as corrections and updates thereto; John D. Roberts and Marjorie C. Caserio (1977) Basic Principles of Organic Chemistry, second edition. W. A. Benjamin, Inc., Menlo Park, Calif.).

### Compositions

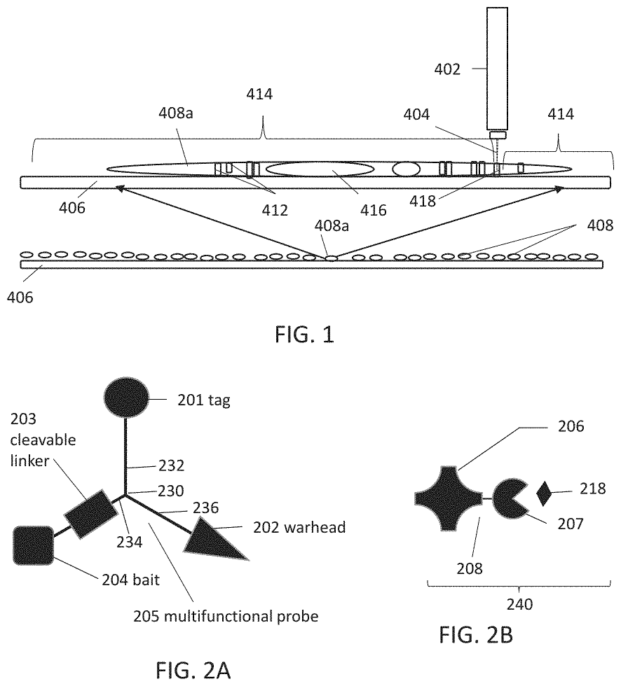

Described herein are compositions of matter including photoreactive and cleavable probes (e.g., bioorthogonally or mildly cleavable probes). The photoreactive and cleavable probes can advantageously be used with a microscope system, such as the systems described herein and in U.S. Patent Publication No. 2018/0367717 A1, to enable automatic labeling of cellular biomolecules proximal to a biomolecule of interest. The labeled molecules may be adjacent the biomolecule of interest or may be close-by but not adjacent, such as when intervening molecules are between the biomolecule of interest and cellular biomolecules for capture or analysis. Molecules that are close-by but not adjacent to a molecule of interest may be part of cell structure or otherwise contribute to a cell microenvironment of interest. FIG. 1 shows a schematic depiction of a system useful for photoselective spatial tagging and labeling. The bottom part of FIG. 1 shows substrate 406, such as a microscope stage, and a monolayer of plurality of cells 408 disposed on the substrate. In some embodiments, the surface of an entire substrate, or a portion of the substrate, can be analyzed using an automated microscope system to identify a region of interest. For example, a sample can be stained or labeled to identify a region of interest. The top part of FIG. 1 shows an expanded view of cell 408a, one of the plurality of cells 408. The cell 408a has a nucleus 416 and a plurality of different types of organelles 412, such as cell membranes, mitochondria, ribosomes, and vacuoles. Microscope system 402 selectively shines narrow band of light 404 onto region of interest (ROI) 418 for analysis of the region of interest 418. The illumination can be selective, and large regions 414 of the cell and substrate are not illuminated. As explained in more detail below, narrow band of light 404 activates a photoreactive and mildly cleavable probe in only the region of interest 418.

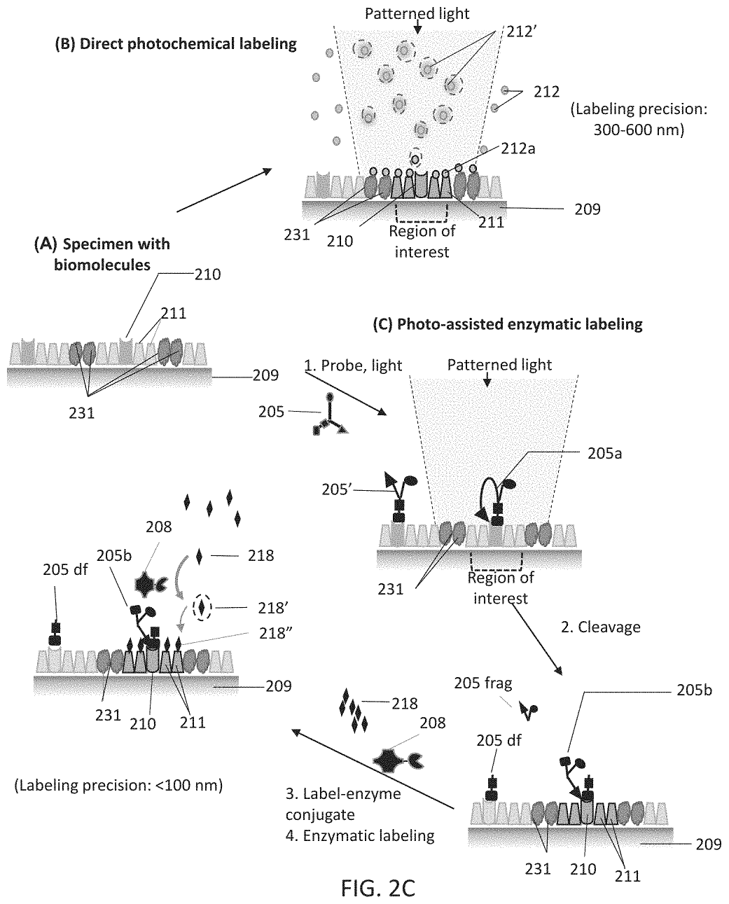

FIG. 2A schematically illustrates multifunctional probe 205 (also referred to interchangeably herein as multifunctional photoreactive and cleavable probe, photoreactive and cleavable probe, or probe unless specific context indicates otherwise). FIG. 2A shows multifunctional probe 205 has a multivalent core 230 with a plurality of attachment sites, first attachment site 232, second attachment site 234, and third attachment site 236. The multifunctional probe of FIG. 2A has tag 201 (circle) attached to first attachment site 232, cleavable linker 203 (rectangle) attached to second attachment site 234, and a light-activated warhead (triangle) attached to third attachment site 236, thus forming a trivalent and trifunctional probe. Bait molecule 204 (rounded square) is attached to cleavable linker 203. FIG. 2B shows an example of a labeling system 240 that can be used with the multifunctional probe 205 shown in FIG. 2B to label biomolecules neighboring a target biomolecule of interest. Labeling system 240 includes labeling complex 208 with label 206 and enzyme or catalyst 207, and enzyme/catalyst substrate 218. In some embodiments, label 206 is NeutrAvidin and enzyme or catalyst 207 is peroxidase and utilizes peroxide (not shown) for activity. In this example, tag 201 and label 206 recognize one another and conjugate. Enzyme or catalyst 207 activates enzyme/catalyst substrate 218 and, once activated, activated enzyme/catalyst substrate 218 can bind to and detectably label biomolecules in its vicinity.

FIG. 2C shows a schematic illustration comparing the results of direct photochemical labeling with photo-assisted enzymatic labeling using the multifunctional photoreactive and cleavable probes described herein to label biomolecules in small region of interest (ROI). FIG. 2B shows a comparison of direct photochemical labeling (top, labeled Process B) and photo-assisted enzymatic labeling (bottom, labeled Process C) using the probes and systems described herein on a specimen with biomolecules (A). Prior to performing either Process B or Process C, a sample (e.g., a cell or tissue sample) containing a biomolecule of interest 210 (protein will be used herein by way of example, but other biomolecules could instead be analyzed) is analyzed and a region of interest identified. The sample can be pretreated, such as fixed and stained. For example, a sample can be fixed and stained with a cell stain (e.g., hematoxylin and eosin (H &E); Masson's trichrome stain), identified with an immunofluorescent labeled antibody recognizing a protein of interest or by other methods. Once the region of interest is identified, a complex of neighboring biomolecules within the region of interest is analyzed. As illustrated in Process B, the sample is treated with a direct photoreactive probe 212 and patterned light is directed to the sample and activate direct photoreactive probe 212 to form activated direct photoreactive probe 212′. The activated direct photoreactive probe 212′ is able to form complexes with other molecules with a close vicinity (show by the dotted circle in Process B. The activated direct photoreactive probe 212′ can diffuse and labels neighbor molecules 211 near the molecule of interest 210. However, the labeling diameter (300-600 nm) of direct photoactivation of photoreactive probes is spatially restricted by the diffraction limit of the light sources used. Additionally, since the photoreactive probe is free to diffuse, any proteins in the pathway of the patterned light can be labeled. Process B also shows it labels more distant biomolecules 231. The region labeled by activated direct photoreactive probe 212′, or labeled precision, covers a region of about 300-600 nm. This region can include biomolecules that are not in close proximity to protein of interest, and in some cases might lead to confusing, misleading or unhelpful results.

In contrast, in Process C, shown on the bottom of FIG. 2C, multifunctional probe 205 preconjugated with bait molecule (see FIG. 2A) recognizing the biomolecule of interest is delivered to the sample on substrate 209. As illustrated in Step 1, patterned light is also directed to the sample. However, here, patterned light activates the photoreactive warhead 202 which binds to molecules or moieties close by. In addition to the light directing a limited region of activation, the photoreactive warhead is constrained by its attachment to the probe 205 and the photoreactive warhead becomes attached to the biomolecule of interest. The attached probe 205a is now double-crosslinked to the biomolecule of interest (or close to it). FIG. 2C also shows Step 2 Cleavage, and the cleavable linker 203 is cleaved, such as by the addition of a protease if the cleavable linked is a cleavable peptide linker. Step 1 and Step 2 also show how background or unwanted labeling is reduced using the probes and methods described herein. In Step 1, a probe 205′ is attached to a biomolecule; however, since the probe 205′ is outside the light delivery region, photoreactive warhead 202 is not activated and does not bind to the biomolecule of interest. In Step 2, the probe 205′ is cleaved into two pieces, fragment 205 frag which is unbound and washed away in a washing step and 205df which is defanged due to removal of tag 201 (which is washed away as part of unbound fragment 205 frag). Neither of the probe fragments 205df or 205 frag are able to label any biomolecules. The probe 205b is cleaved, but remains attached to the biomolecule of interest by double-crosslinking. In some variations, the probe 205c may be crosslinked to a bait molecule or another proximal biomolecule; however, the principle remains the same. The probe 205b contains tag 201, and as explained in more detail below, labels neighbor molecules.

Labeling system 240 includes labeling complex 208 with label 206 and enzyme or catalyst 207, and enzyme/catalyst substrate 218.

Excess probe is washed away with wash solution and single-crosslinked probes (e.g., in non-lighted areas) are removed through site-specific cleavage as described above. Steps 3 and 4 show labeling of the molecules near the molecule of interest 210 using labeling system 240 shown in FIG. 2B. Other labeling systems can also be used. By way of example, complex 208 conjugates with tag 201, the enzyme or catalyst 207 activates enzyme/catalyst substrate 218 to activated enzyme/catalyst substrate 218′. Since probe 205b is attached to molecule of interest 210, neighbor molecules 211 are labeled, while more distant molecule 231 is not. The cleavable linkers described herein can enable label transfer from the probe to neighbor molecules within a radius of <10 nm (referring to the size of the radius of the trifunctional (multifunctional) probe).

By photoselectively localizing enzyme or catalyst 207, such as peroxidase, near the molecule of interest and labeling the neighbor molecules 211 in the region of interest using the tagging and labeling just described, the coupling reaction can be localized to a region as small as <100 nm. In some variations, a larger region (e.g., up to about 200 nm, up to about 300 nm, up to about 400 nm) could be labeled. Furthermore, some molecules of interest in a sample have more one region of localization and hence interact with different molecular complexes in different locations simultaneously. The light-assisted tag transfer (e.g., tagging neighbor molecules) can be used successively in more than one location. For example, after applying light as shown in FIG. 2B Process C and tagging the neighbor molecules as indicated, the light can be selectively applied to a second (third, fourth, etc.) location in the sample and this process can be repeated as many times as desired. In addition to labeling (depositing labels) to a relatively small number of neighbor molecules in a very small area of a sample, such as due to the use of the microscope analysis to direct the light and the probes described herein, and as explained below, the process can also be performed with sufficiently mild or gentle treatments so that the cell architecture remains intact (e.g., the reactions are also bioorthogonal).

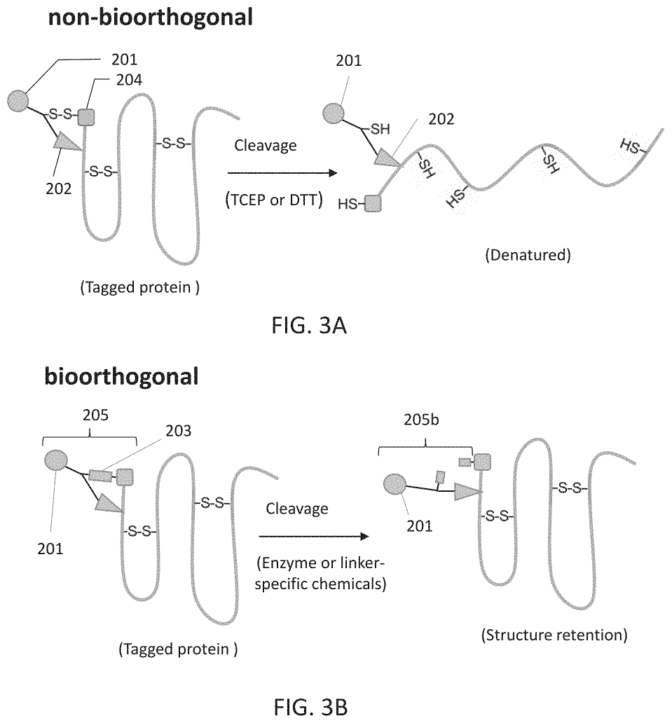

FIG. 3A and FIG. 3B schematically illustrate the effects on protein structure using a multifunctional photoreactive and cleavable probe and mild cleavage conditions as described herein (FIG. 3B), compared with results using a probe with harsh cleavage reactions (FIG. 3A). With mild cleavage conditions the protein structure is retained and the reaction is bioorthogonal, while with harsh cleavage conditions, the protein denatures and the reaction is non-bioorthogonal. FIG. 3A schematically illustrates a relatively harsh cleavage, such as one mediated by use of a reducing agent such as tris (2-carboxyethyl) phosphine (TCEP) or dithiothreitol (DTT) in a cleavage reaction. In addition to cleaving the linker, TCEP or DTT break other disulfide bonds, including naturally occurring covalent disulfide bonds commonly found between cysteine amino acids in proteins, denaturing the proteins. It has been estimated that more than 90% of proteins in cells contain at least one cysteine amino acid and that some one-third of the proteins in the eukaryotic proteome form disulfide bonds. Thus, performing a relatively harsh cleavage on a sample to break disulfide bonds is likely to significantly disrupt protein structure, disrupt overall cell architecture, and alter naturally occurring biomolecule interactions. The cleavage reaction can be considered to be a non-bioorthogonal reaction. In some embodiments, a bioorthogonal reaction preserves structures derived from living organisms (e.g., derived from eukaryotes) and excludes consideration of non-living entity structures, such as viruses.

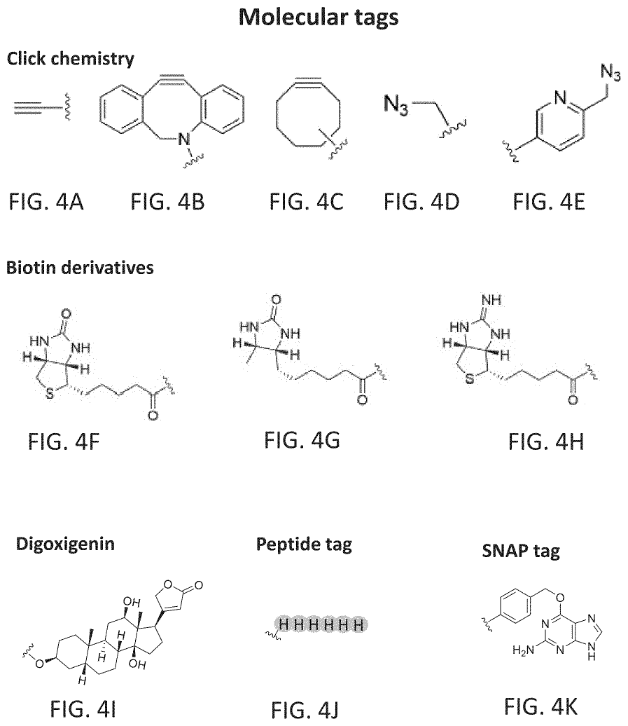

FIG. 4B schematically illustrates a relatively mild cleavage reaction for use with the multifunctional probes described herein. The cleavage reaction uses gentler reagents, such as enzymes or linker-specific chemicals, to cleave the cleavable linker. In some embodiments, mild cleavage reagents are substantially specific. In other words, they substantially and specifically bind to and cleave targets of interest (e.g., the cleavable linker), while substantially not binding to or cleaving other molecules (e.g., less than 1% of the time, less than 0.1%, etc.). In some embodiments, mild cleavage reagents act to cleave other bonds, such as C—C bonds and leave bonds such as disulfide (—S—S—) bonds intact. As illustrated in FIG. 3B, the three-dimensional structure of the protein, mediated by disulfide bonds, remains intact after mild cleavage as described herein. Since tagging and proximity labelling of, for example, naturally occurring neighboring molecules neighboring a protein of interest depends upon the relative proximity of the neighboring molecules to the protein of interest, maintaining the three-dimensional structure of biomolecules and the overall cell architecture can lead to more accurate tagging and labeling of neighboring molecules, reducing both false positives and false negatives in a mild cleavage reaction. The mild cleavage reaction can be bioorthogonal in that it does not substantially disrupt naturally occurring protein structure or cell architecture.

FIGS. 4A-4K show examples of tags that can be used in the photoreactive and cleavable probes described herein. The tags are configured to interact with a detectable label to label biomolecules neighboring a target molecule of interest. FIG. 4A-FIG. 4E show examples of click chemistry tags that can be used with the probes. The click chemistry tag may be, for example, an azide moiety or an alkyne moiety. FIGS. 4F-4H show examples of biotin derivatives that can be used with as probe tags. FIG. 4I shows a digoxigenin moiety tag. FIG. 4J shows a peptide tag. In particular, FIG. 4J shows a poly His tag with 6 histidines (SEQ ID NO:1). However, a histidine tag could instead fewer or more histidines, such as 5 (SEQ ID NO: 42) or 7-10 or more (SEQ ID NO: 43). FIG. 4K shows a SNAP-tag. FIG. 6K shows a SNAP-tag and a CLIP-tag or HaloTag could also be used.

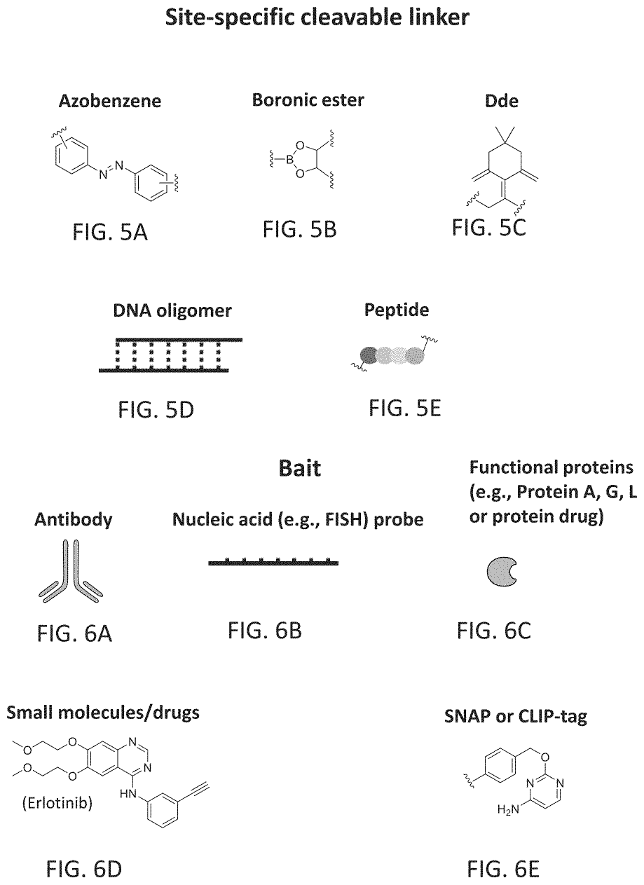

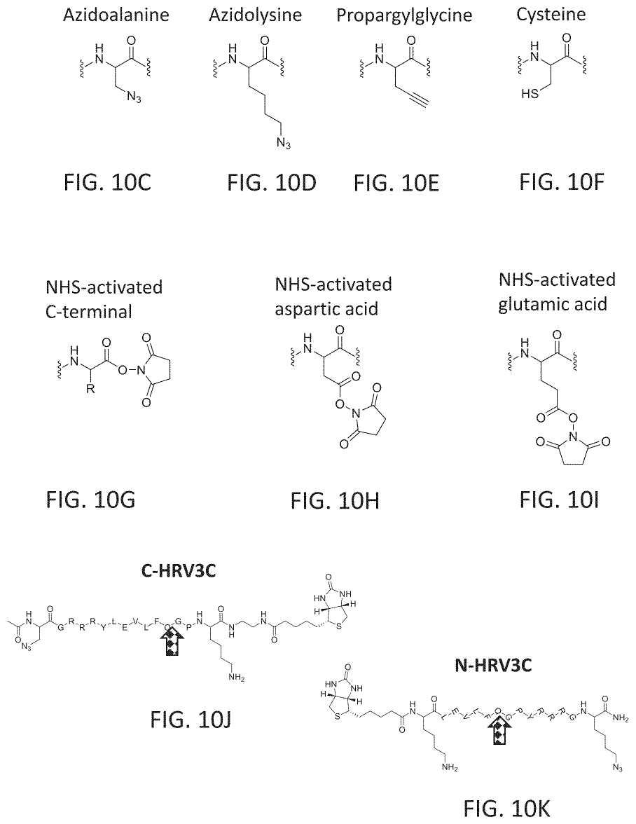

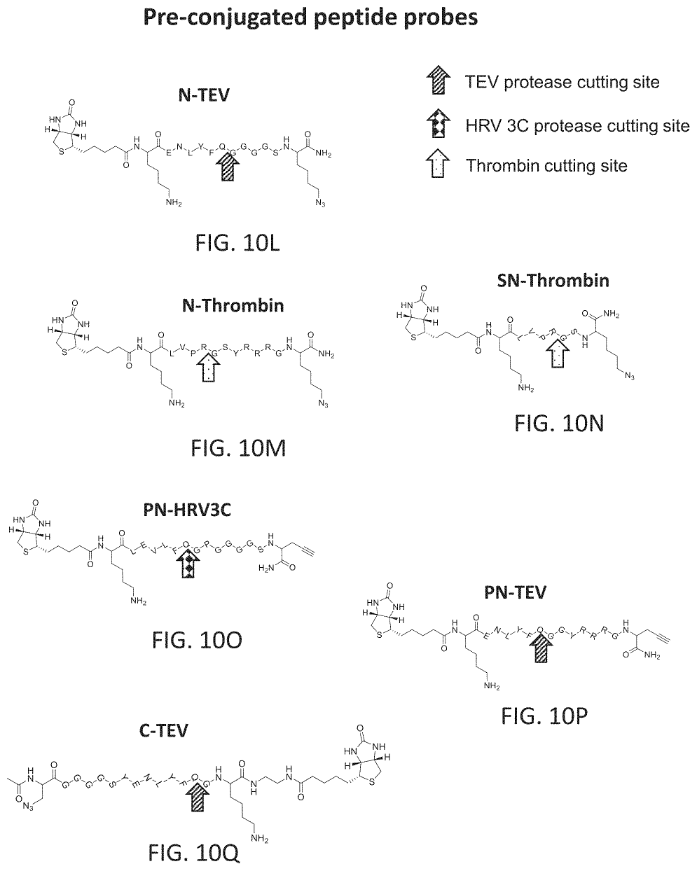

FIGS. 5A-5E show examples of site-specific cleavable linkers that can be used in the photoreactive and cleavable probes described herein. FIG. 5A shows an azobenzene moiety. An azobenzene linker can be cleaved during the cleavage step such as with sodium dithionite or azoreductase. FIG. 5B shows a boronic ester moiety. A boronic ester cleavable linker can be cleaved with thionyl chloride and pyridine. FIG. 5C shows a Dde moiety. The Dde cleavable linker can be cleaved using enzymes or simple small molecules. FIG. 5D shows a DNA oligomer cleavable linker and other nucleic acid molecules can instead be used. DNA oligomers can be cleaved using restriction enzymes, nucleases, or competitive methods using complementary oligomers, depending upon what molecule is labeled. FIG. 5E shows a peptide moiety linker and peptide moiety linkers are discussed below in more detail in reference to FIG. 10A-FIG. 10Q. A peptide linker can be cleaved during the cleavage step using a protease. In some embodiments, a site-specific cleavable linker can be conjugated to a bait molecule. For example, a linker conjugating to bait molecules such as NETS-esters can bind to protein baits, such as antibodies. A particular cleavage linker and associated cleavage reagent can be chosen for various reasons, such as cost or cleavage efficiency.

FIGS. 6A-6E shows examples of bait molecules that can be used in the photoreactive and cleavable probes described herein to conjugate with a molecule of interest in a sample. FIG. 6A shows an antibody that can be used a bait molecule. Any time of antibody can be used. FIG. 6B shows a nucleic acid portion that can be used as a bait molecule, such as fluorescent in situ hybridization probe (FISH probe). FIG. 6C shows a representation of a functional protein that can be used as a bait molecule. Examples of functional proteins include Protein A, Protein G, Protein L, protein A/G, or a protein drug. Other bait molecules that can be used in the photoreactive and cleavable probes described herein include biologic drugs. Examples of biologic drugs that can be used as bait include abatacept (Orencia); abciximab (ReoPro); abobotulinumtoxinA (Dysport); adalimumab (Humira); adalimumab-atto (Amjevita); ado-trastuzumab emtansine (Kadcyla); aflibercept (Eylea); agalsidase beta (Fabrazyme); albiglutide (Tanzeum); aldesleukin (Proleukin); alemtuzumab (Campath, Lemtrada); alglucosidase alfa (Myozyme, Lumizyme); alirocumab (Praluent); alteplase, cathflo activase (Activase); anakinra (Kineret); asfotase alfa (Strensiq); asparaginase (Elspar); asparaginase Erwinia chrysanthemi (Erwinaze); atezolizumab (Tecentriq); basiliximab (Simulect); becaplermin (Regranex); belatacept (Nulojix); belimumab (Benlysta); bevacizumab (Avastin); bezlotoxumab (Zinplava); blinatumomab (Blincyto); brentuximab vedotin (Adcetris); canakinumab (Ilaris); capromab pendetide (ProstaScint); certolizumab pegol (Cimzia); cetuximab (Erbitux); collagenase (Santyl); collagenase clostridium histolyticum (Xiaflex); daclizumab (Zenapax); daclizumab (Zinbryta); daratumumab (Darzalex); darbepoetin alfa (Aranesp); denileukin diftitox (Ontak); denosumab (Prolia, Xgeva); dinutuximab (Unituxin); dornase alfa (Pulmozyme); dulaglutide (Trulicity); ecallantide (Kalbitor); eculizumab (Soliris); elosulfase alfa (Vimizim); elotuzumab (Empliciti); epoetin alfa (Epogen/Procrit); etanercept (Enbrel); etanercept-szzs (Erelzi); evolocumab (Repatha); filgrastim (Neupogen); filgrastim-sndz (Zarxio); follitropin alpha (Gonal f); galsulfase (Naglazyme); glucarpidase (Voraxaze); golimumab (Simponi); golimumab injection (Simponi Aria); ibritumomab tiuxetan (Zevalin); idarucizumab (Praxbind); idursulfase (Elaprase); incobotulinumtoxinA (Xeomin); infliximab (Remicade); infliximab-dyyb (Inflectra); interferon alfa-2b (Intron A); interferon alfa-n3 (Alferon N Injection); interferon beta-1a (Avonex, Rebif); interferon beta-1b (Betaseron, Extavia); interferon gamma-1b (Actimmune); ipilimumab (Yervoy); ixekizumab (Taltz); laronidase (Aldurazyme); mepolizumab (Nucala); methoxy polyethylene glycol-epoetin beta (Mircera); metreleptin (Myalept); natalizumab (Tysabri); necitumumab (Portrazza); nivolumab (Opdivo); obiltoxaximab (Anthim); obinutuzumab (Gazyva); ocriplasmin (Jetrea); ofatumumab (Arzerra); olaratumab (Lartruvo); omalizumab (Xolair); onabotulinumtoxinA (Botox); oprelvekin (Neumega); palifermin (Kepivance); palivizumab (Synagis); panitumumab (Vectibix); parathyroid hormone (Natpara); pegaspargase (Oncaspar); pegfilgrastim (Neulasta); peginterferon alfa-2a (Pegasys); peginterferon alfa-2b (PegIntron, Sylatron); peginterferon beta-1a (Plegridy); pegloticase (Krystexxa); pembrolizumab (Keytruda); pertuzumab (Perjeta); ramucirumab (Cyramza); ranibizumab (Lucentis); rasburicase (Elitek); raxibacumabreslizumab (Cinqair); reteplase (Retavase); rilonacept (Arcalyst); rimabotulinumtoxinB (Myobloc); rituximab (Rituxan); romiplostim (Nplate); sargramostim (Leukine); sebelipase alfa (Kanuma); secukinumab (Cosentyx); siltuximab (Sylvant); tbo-filgrastim (Granix); tenecteplase (TNKase); tocilizumab (Actemra); trastuzumab (Herceptin); ustekinumab (Stelara); vedolizumab (Entyvio); ziv-aflibercept (Zaltrap).

FIG. 6D also shows small molecules/drugs can be used as bait molecules. By way of example, erlotinib is shown. FIG. 6E shows a CLIP-tag and other members of self-labeling moieties could be used (e.g., HaloTag or SNAP-Tag).

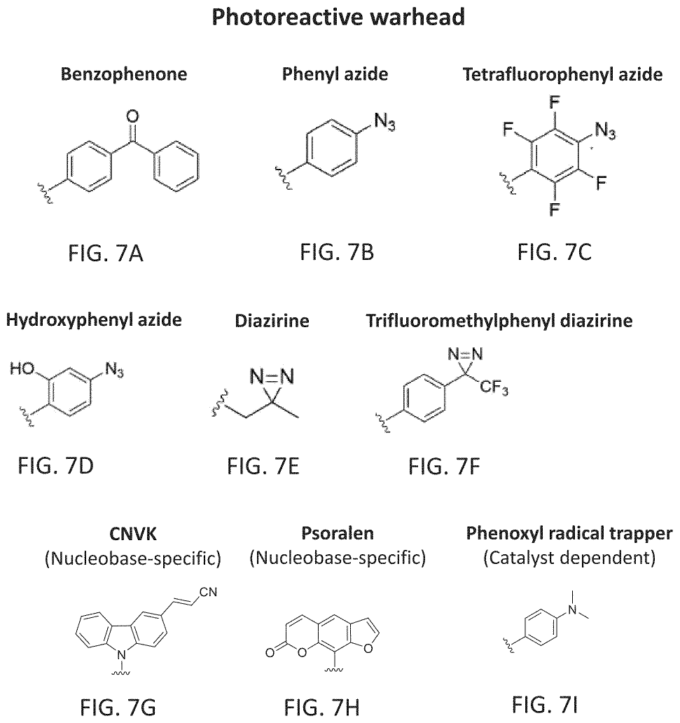

FIGS. 7A-7I show examples of photoactive warheads that can be used in the photoreactive and cleavable probes described herein. FIG. 7A shows a benzophenone photoactive warhead, which can be activated by either 320-365 nm UV-A irradiation of single photon excitation or 720-800 nm of two photon excitation. FIGS. 7B, 7C and 7D shows aryl azide-based warheads which can be activated by either 250-365 nm irradiation of single photon excitation or 800 nm of two photon excitation. FIG. 7B shows phenyl azide photoactive warheads. FIG. 7C shows tetrafluorophenyl azide photoactive warheads. FIG. 7D shows hydroxyphenyl azide photoactive warheads. FIG. 7E shows diazirine photoactive warheads. FIG. 7F shows trifluoromethylphenyl diazirine photoactive warheads. FIG. 7G shows 3-cyanovinylcarbazole nucleoside (CNVK) photoactive warheads which is nucleobase specific. FIG. 7H shows psoralen photoactive warheads which is also nucleobase specific. Psoralens react with DNA or RNA to form covalent adducts. In some embodiments, psoralen photoactive warheads can be activated by long wavelength US light (e.g., UVA, 310-400 nm). FIG. 7I shows phenoxyl radical trapper photoactive warheads which is catalyst dependent. The selection of a particular light-activated warhead can depend on the desired wavelength and the types of the bait molecule. For example, the constituents of the multifunctional probe and constituents for the pre-probe analysis can be chosen so as to not interfere (or minimally interfere) with each other.

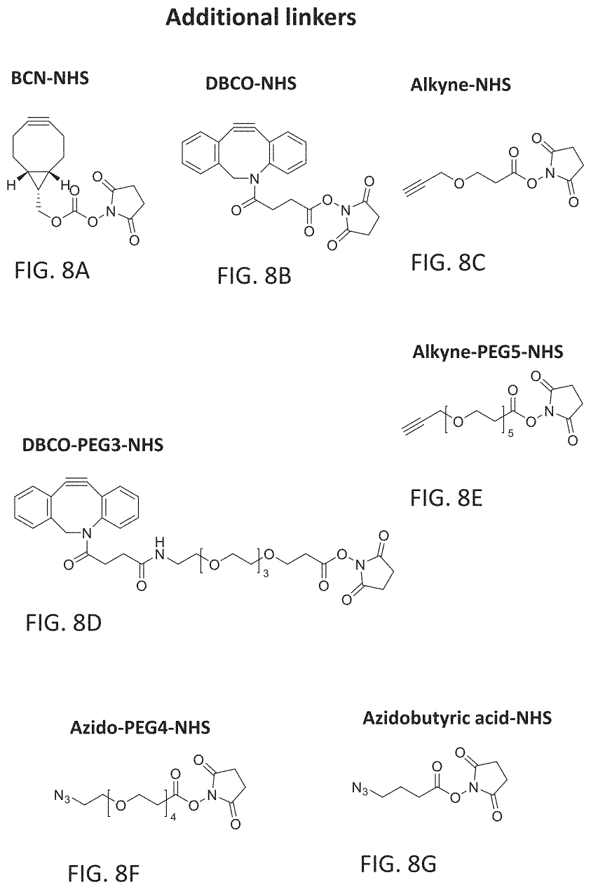

FIG. 8A-8G show additional examples of linkers that can be used as linkers in the photoreactive and cleavable probe described herein. FIG. 8A shows a BCN-NHS linker. FIG. 8B shows DBCO-NHS linker. FIG. 8C shows Alkyne-NHS linker. FIG. 8D shows DBCO-PEG3-NHS linker. FIG. 8E shows Alkyne-PEG5-NHS linker. FIG. 8F shows Azido-PEG4-NHS linker. FIG. 8G shows azidobutyric acid-NETS linker.

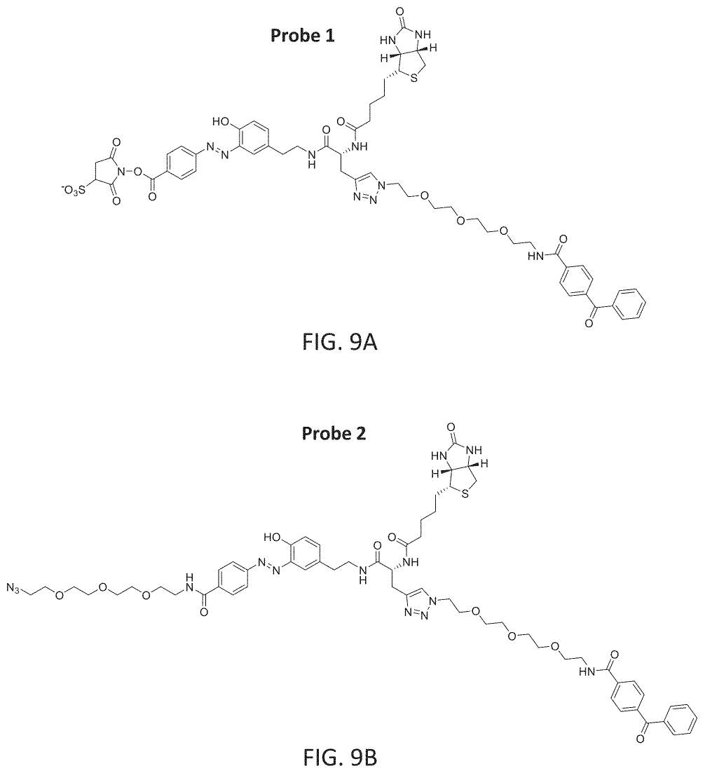

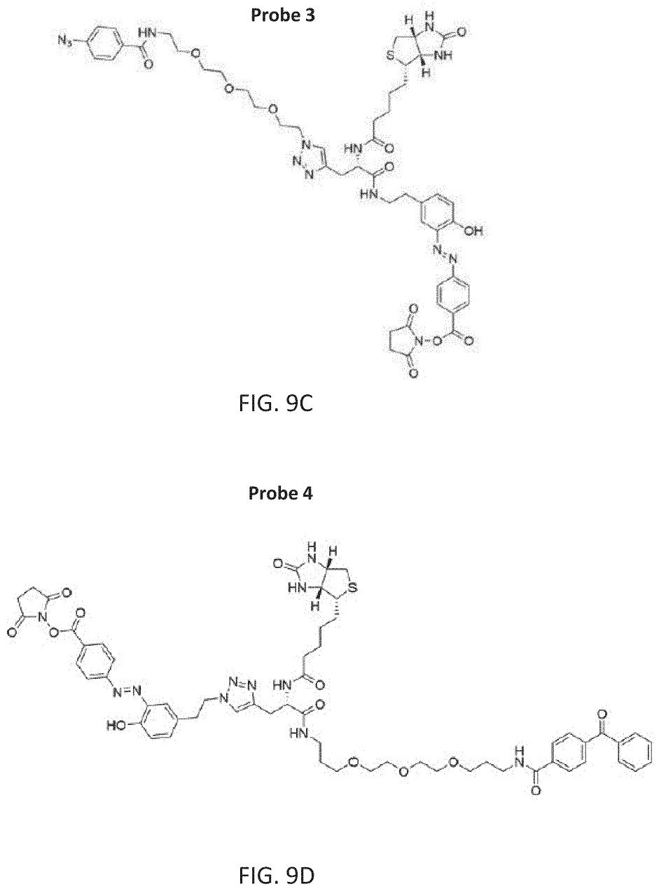

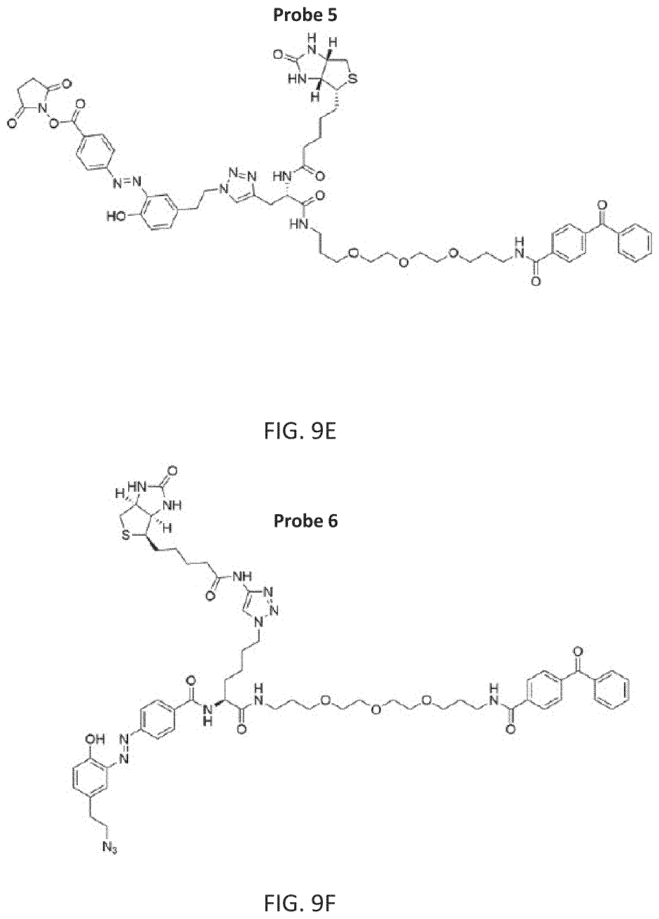

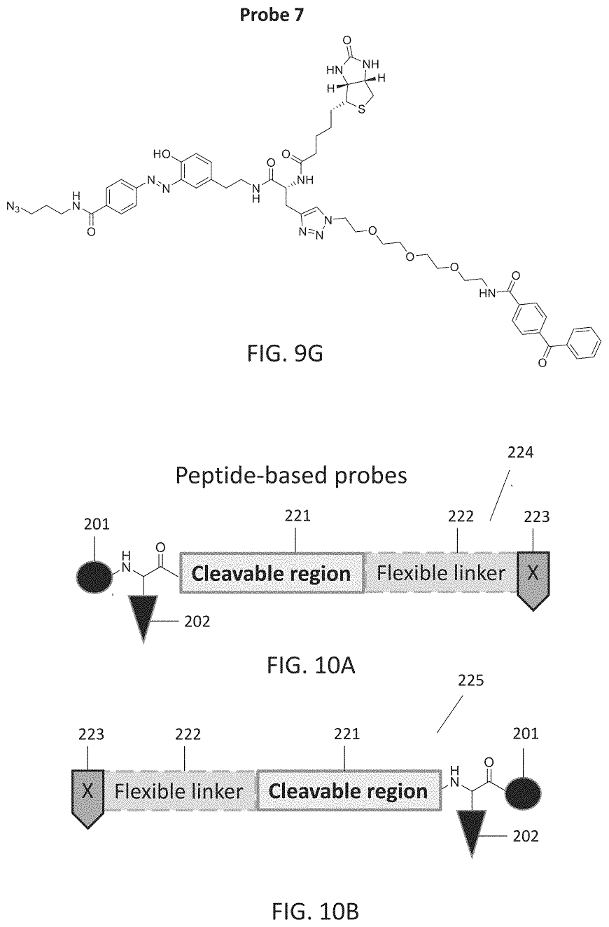

FIGS. 9A-9G show examples of photoreactive and cleavable probes that can be used in the compositions and for practicing the methods described herein. The probes have multivalent cores with a plurality of attachment sites. A tag, a cleavable linker, and a light-activated warhead are bound to the attachment sites on the probe. In some embodiments, the multivalent core includes the moiety of formula (I). In some embodiments, n is 1, 2, 3, 4, 5, or 6. In some embodiments, R1 and R2 each independently are hydrogen, substituted, alkyl, substituted alkenyl, substituted alkynyl, substituted carbocyclyl, substituted heterocyclyl, substituted aryl, substituted heteroaryl, or a nitrogen protecting group. In some embodiments one of R3 and R4 is —(CH2)x(OCH2CH2)y(CH2)zNR5R6, and the other is an attachment site, wherein x is 1, 2, 3, 4, 5, or 6; y is 1, 2, 3, 4, 5, or 6; z is 0, 1, 2, 3, 4, 5, or 6; and one of R5 and R6 is an attachment site, and the other is hydrogen, substituted alkyl, substituted alkenyl, substituted alkynyl, substituted carbocyclyl, substituted heterocyclyl, substituted aryl, substituted heteroaryl, or a nitrogen protecting group.

FIGS. 10A-10B schematically illustrate peptide-based photoreactive and cleavable probes. These probes have a peptide region cleavable by a peptide cleavage reagent, such as by a protease that recognizes a specific peptide sequence and the peptide regions are specifically cleavable (e.g., by a protease). FIG. 10A shows an example of a peptide-based probe 224 with tag 201 and photoreactive warhead 202 on the N-terminal end of the peptide region. FIG. 10B shows an example of a peptide-based probe 225 with tag 201 and warhead 202 on the C-terminal end of the peptide region. FIGS. 10A-10B also show probes with an additional, flexible linker 222 (also referred to herein as a spacer) and an optional clickable amino acid 223. FIGS. 10C-10I show examples of reactive or clickable amino acids that can be used with the probes shown in FIGS. 10A and 10B. FIG. 10C shows azidoalanin clickable amino acid. FIG. 10D shows azidolysine clickable amino acid. FIG. 10E shows propargylglycine clickable amino acid. FIG. 10F shows cysteine clickable amino acid. FIG. 10 G shows NETS-activated C-terminal clickable amino acid. FIG. 10H shows NETS-activated aspartic acid clickable amino acid. FIG. 10I shows NETS-activated glutamic acid.

FIGS. 10J-10Q show examples of peptide-based photoreactive and cleavable probes schematically illustrated in FIGS. 10A-10B. The cleavage sites for the human rhinovirus 3C (HRV 3C) protease (checkered arrow), tobacco etch virus (TEV) protease (striped arrow), and thrombin (dotted arrow) are indicated. The proteolytically cleavable peptide sequences can be specifically cleaved by a protease during the cleavage step. Examples of proteolytically cleavable peptide sequences that can be used with the probes described herein include those recognized by activated blood coagulation factor X enteropeptidase (also referred to herein as factor X enteropeptidase or factor Xa), human rhinovirus (HRV) 3C protease, thrombin, and tobacco etch virus (TEV) protease. Of these proteases, the factor Xa and thrombin are naturally found in blood. These proteases may recognize and cleave proteins in a cell or cell extract other than the proteolytically cleavable peptide sequences of the peptide-based probes. Although in some cases, this may upset the naturally occurring protein environment in the samples and lead to misleading or artefactual results in some analyses, it is also noted that the number of these cleavage reactions may be sufficiently limited so as to be useful for certain purpose or in certain situations. Cleavage reactions that do not interfere with naturally occurring biomolecules (e.g., naturally occurring proteins in a cell or tissue sample) are considered bioorthogonal and probes cleavable under circumstances that maintain naturally occurring protein structure can be considered to be a bioorthogonally cleavable probe with a bioorthogonally cleavable peptide sequence. As discussed above, while the Sulfo-SBED probe may find use with certain of the methods described herein for particular applications, in other embodiments, the cleavage of the Sulfo-SBED probe with dithiothreitol (DTT) or 2-mercaptoethanol to cleave its S—S bond also undesirably disrupts naturally occurring proteins (e.g., it is non-bioorthogonal).

Enterokinase recognizes the peptide sequence DDDDK| (SEQ ID NO: 2) for cleavage, where cleavage occurs in the linker bond after the lysine.

Factor Xa recognizes the peptide sequence LVPR|GS (SEQ ID NO:3) for cleavage, where cleavage occurs in the linker bond between the arginine and the glycine.

Human rhinovirus (HRV) 3C protease recognizes the peptide sequence LEVLFQ|GP (SEQ ID NO:4) for cleavage, where cleavage occurs in the linker bond between the glutamine and the glycine.

TEV protease prefers the peptide sequence ENLYFQ|S (SEQ ID NO:5) for cleavage, where cleavage occurs in the linker bond between the glutamine and the serine. TEV protease can also recognize the sequence ENLYFQ|G (SEQ ID NO:6) for cleavage, where cleavage occurs between the linker bond between the glutamine and the glycine.

Thrombin recognizes the peptide sequence LVPR|GS (SEQ ID NO:7) for cleavage, where cleavage occurs in the linker bond between the arginine and the glycine

In addition to specific recognition sequences for proteolysis, the peptide portion may contain additional amino acids. Photoreactive and cleavable probes can have either C-terminal or N-terminal tags (e.g., biotinylation).

FIG. 10J shows a C-HRV3C pre-conjugated peptide probe with an HRV 3C proteolytically cleavable peptide sequences GRRRYLEVLFQGP (SEQ ID NO: 8).

FIG. 10K shows an N-HRV3C pre-conjugated peptide probe with an HRV 3C protease cutting site peptide sequence LEVLFQGPYRRRG (SEQ ID NO: 9).

FIG. 10L shows an N-TEV pre-conjugated peptide probe with a TEV protease cutting site peptide sequence ENLYFQGGGGS (SEQ ID NO: 10).

FIG. 10M shows an N-Thrombin pre-conjugated peptide probe with a thrombin protease cutting site peptide sequence LVPRGSYRRRG (SEQ ID NO: 11).

FIG. 10N shows SN-Thrombin conjugated peptide probe with a thrombin protease cutting site peptide sequence LVPRGS (SEQ ID NO: 12).

FIG. 10O shows PN-HRV3C conjugated peptide probe with HRV 3C protease cutting site peptide sequence LEVLFQGPGGGGS (SEQ ID NO: 13).

FIG. 10P shows a PN-TEV conjugated peptide probe with a TEV protease cutting site peptide sequence ENLYFQGGYRRRG (SEQ ID NO: 14).

FIG. 10Q shows a C-TEV conjugated peptide probe with a TEV protease cutting site peptide sequence GGGGSYENLYFQG (SEQ ID NO: 15).

As indicated above, some probes include a flexible linker (also referred to herein as a spacer). Flexible linkers are flexible molecules or stretches of molecules that are used to link two molecules or moieties together. Linkers may be composed of flexible groups so that adjacent domains are free to move relative to another. Flexible linkers may include flexible amino acid residues, such as glycine (G) or serine (S). Flexible linkers may also include threonine (T) and alanine (A) residues. A string of amino acids can be repeated in the linker. For example, a linker may include a length of glycine residues followed by a serine residue, such as forming an (GGGGS)n oligomer, where n is 1, 2, 3, 4, 5, 6, 7, 8 or larger (SEQ ID NO: 22) and the GGGGS motif (SEQ ID NO: 23) is repeated. Flexible linkers can also include alkyl groups, such as a polyethylene glycol (CH₂CH₂O)m linker, where m is from 1 to 50, or 2-30, or 3-6. Other examples of polymeric flexible linkers include polypropylene glycol, polyethylene, polypropylene, polyamides, and polyesters. Flexible linkers can be linear molecules in a chain of at least one or two atoms and can include more.

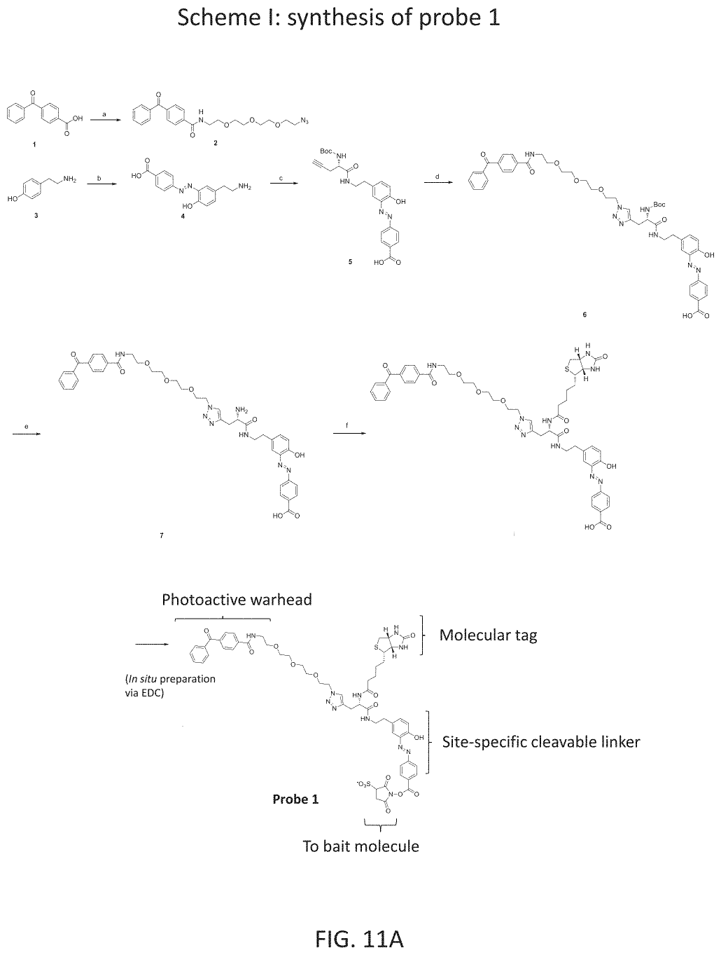

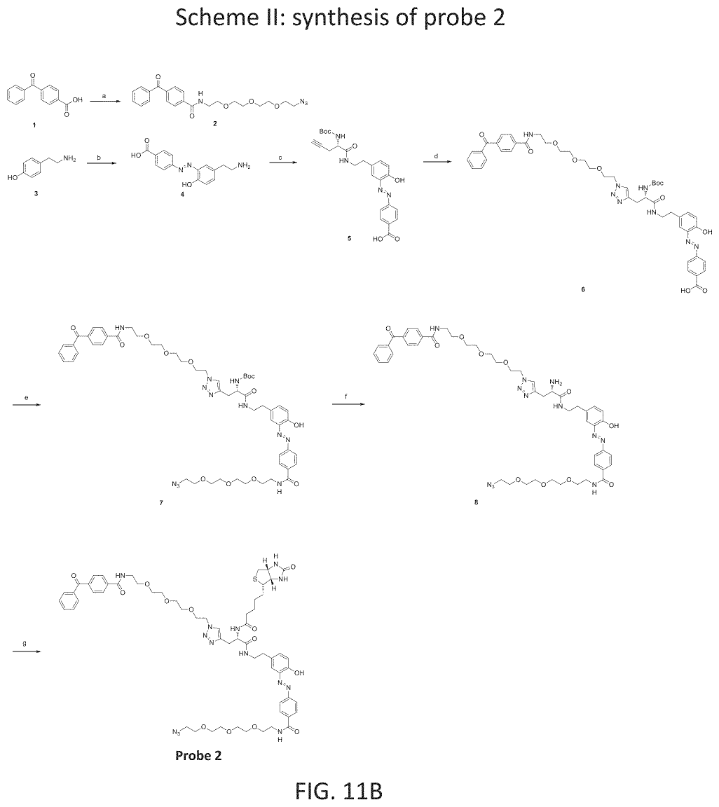

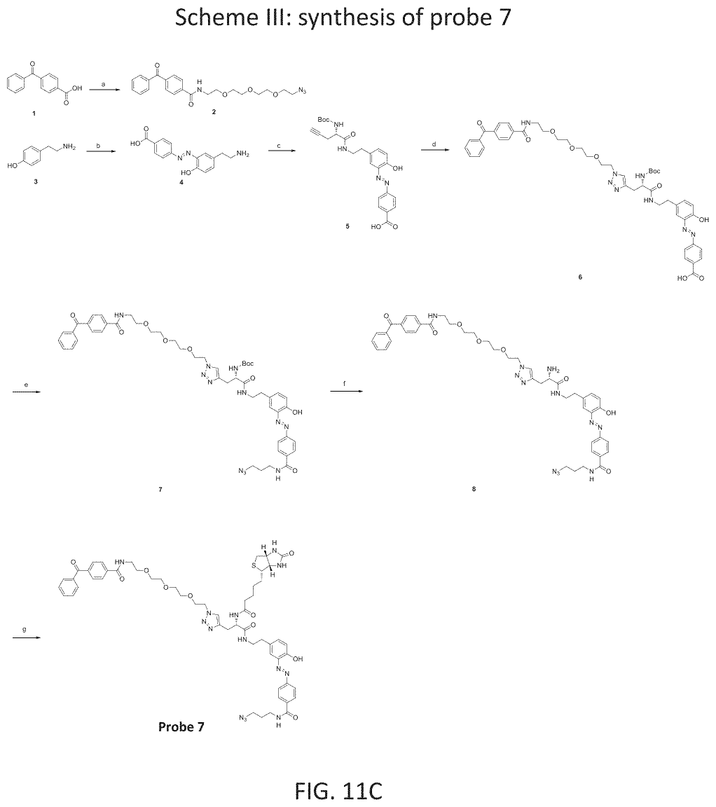

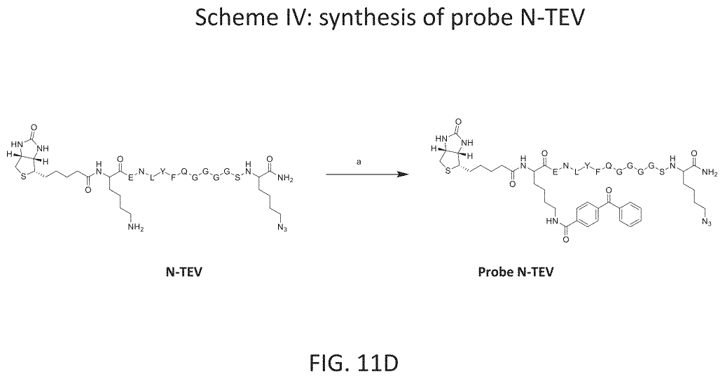

FIGS. 11A-11D illustrate methods to synthesize the photoreactive and cleavable probes described herein. The methods create probes with a tag, a cleavable linker, and a light-activated warhead. The figures also illustrate regions where bait molecules can be conjugated. The trifunctional molecular probes can be synthesized by using commercially available molecules as building blocks and regular-used synthesis steps. The schemes shown in FIGS. 11A-11D for synthesis of the probes are given as examples and not for limiting purposes. FIG. 11A shows a synthesis scheme for probe 1. FIG. 11B shows a synthesis scheme for probe 2. FIG. 11C shows a synthesis scheme for probe 7. FIG. 11D shows a synthesis scheme for Probe IV N-TEV.

Some embodiments provide a photoreactive and cleavable probe including a multivalent core comprising a plurality of attachment sites. Some embodiments provide a tag bound to one of the attachment sites, wherein the tag is configured to conjugate to a label. Some embodiments provide a cleavable linker bound to a second of the attachment sites and configured to link to a bait molecule, wherein the cleavable linker includes a peptide sequence. Some embodiments provide a light-activated warhead bound to a third of the attachment sites, wherein the multivalent core includes the moiety of formula (II) or (III):

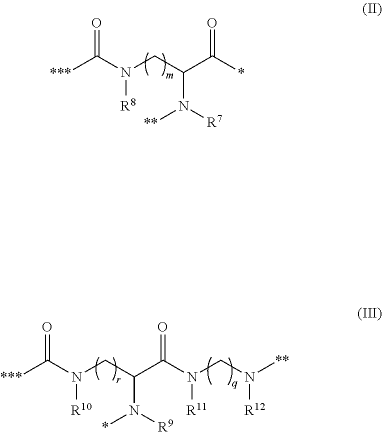

wherein m, r and q each independently are 1, 2, 3, 4, 5, or 6; wherein * comprises an attachment site of one of the plurality of attachment sites for the cleavable linker, wherein ** includes a different attachment site of the plurality of attachment sites for one of either the tag or the photoreactive warhead; wherein *** includes a different attachment site of the plurality of attachment sites for either the photoreactive warhead or the tag, respectively, and R7, R8, R9, R10, R11, and R12 each independently are hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, or a nitrogen protecting group.

In some embodiments of the photoreactive and cleavable probe wherein ** includes the attachment site for the tag, and *** includes the attachment site for the photoreactive warhead.

In some embodiments of the photoreactive and cleavable probe, the peptide sequence includes a protease recognition sequence.

In some embodiments of the photoreactive and cleavable probe, wherein the peptide sequence comprises a human rhinovirus 3C (HRV 3C) protease recognition sequence, a tobacco etch virus (TEV) protease recognition sequence, or a thrombin recognition sequence.

In some embodiments of the photoreactive and cleavable probe, the cleavable linker further includes a conjugatable amino acid.

In some embodiments of the photoreactive and cleavable probe, the cleavable linker further includes a cysteine or clickable amino acid amino acid.

In some embodiments of the photoreactive and cleavable probe, the cleavable linker comprises a clickable amino acid with an azido or alkyne moiety.

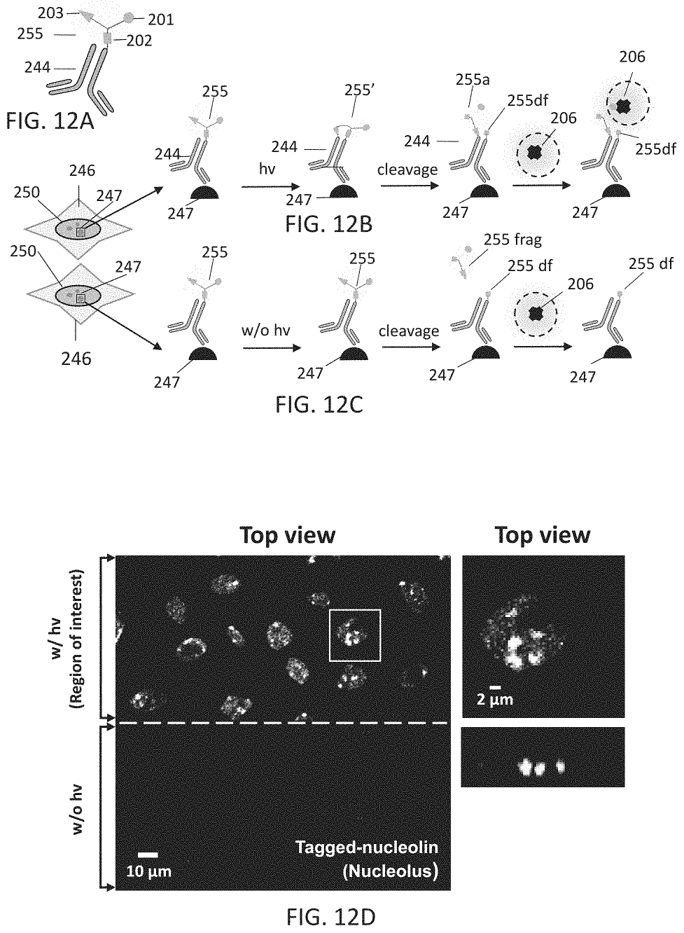

FIG. 12A schematically illustrates a photoreactive and cleavable probe conjugated to an antibody bait. FIG. 12B and FIG. 12C schematically illustrates a reaction scheme for performing photoselective tagging of a molecule using a photoreactive and cleavable probe conjugated to an antibody bait for labeling proteins in the cell nucleolus. FIG. 12B illustrates how the reaction proceeds using controlled light. FIG. 12B also illustrates how the cleavable probes are cleaved to reduce background in non-lighted areas. The reaction shown in FIG. 12B is similar as to that shown in FIG. 2C except that bait molecule 204 is an antibody 244 and the probe 255 includes the antibody 244 as bait. When the photoreactive warhead of the probe 255 is activate, it binds to the bait/antibody 244, rather than a cellular molecule. However, the probe 255 is still retained in the vicinity of the protein of interest, in this case nucleolin in nucleolus 247 in the nucleus 250 of cell 246. During the cleavage step, probe 255 out of the photoselected area is still cleaved into fragment 255 frag and defanged probe fragment 255df which are washed away during a wash step. FIG. 12D shows results from using the reaction schemes shown in FIG. 12A and FIG. 12B. The nucleolin protein is specifically tagged in the presence of light (top and right panels) but is not tagged in the absence of light (bottom panel). Probes linked to bait molecules are selectively retained through light-activation followed by cleavage and conjugated to enzymes (e.g., HRP in this example) for spatial labeling at a radius from about 10 nm to about 100 nm, depending upon the particular enzymes and reaction times used. In some embodiments of the method, selectively illuminating includes illuminating a zone defined by point spread function.

Photoselective tagging and labeling as described herein can be performed in various types of samples, such as samples obtained from tissues, cells, or particles, such as from an entity (e.g., a human subject, a mouse subject, a rat subject, an insect subject, a plant, a fungi, a microorganism, a virus) or tissues samples or cell samples that are not from an organism, such as cell culture samples or artificial tissue scaffold samples (e.g., cultured laboratory cells, in vitro developed heart tissue, 3-d printed tissue, etc.). Samples for analysis using the probes, materials, and methods described herein can be living (live cells) or can be not living (e.g., fixed). A sample for tagging and layering can include a monolayer sample, a multi-layer sample, a sample fixed to a substrate (e.g., a microscope slide), a sample not fixed to a substrate, a suspension of cells, or an extract, such as an in vitro cell extract, a reconstituted cell extract, or a synthetic extract. In some embodiments, a sample is not fixed (unfixed). Examples of probes useful for tagging live cells include those utilizing a small molecule or those sometimes referred to as self-labeling molecules (e.g., Clip-tag, Halo-tag, SNAP-tag). In some embodiments a large number of cells can be automatically analyzed using the methods and materials described herein (e.g., at least about 1,000 cells, at least 10,000 cells, at least 100,000 cells, at least 1 million cells). In some embodiments, a smaller number of cells can be analyzed, such as no more than 1,000 cells, no more than 100 cells, or only a few cells or a single cell. In some embodiments a sample is fixed. For example, a cell or tissue sample may be fixed with e.g., acetic acid, acetone, formaldehyde (4%), formalin (10%), methanol, glutaraldehyde, or picric acid. A fixative may be a relatively strong fixative and may crosslink molecules or may be weaker and not crosslink molecules. A cell or tissue sample for analysis may be frozen, such as using dry ice or flash frozen, prior to analysis. A cell or tissue sample may be embedded in a solid material or semi-solid material such as paraffin or resin prior to analysis. In some embodiments, a cell or tissue sample for analysis may be subject to fixation followed by embedding, such as formalin fixation and paraffin embedding (FFPE).

In some variations, a functional group that facilitates the interaction of a warhead with its target can be utilized. A functional group configured to facilitate interaction between a warhead and its target can be incorporated into a probe, target (e.g., into a molecule, such as a carbohydrate, lipid, nucleic acid, or protein), or another molecule. Such a functional group may, for example, facilitate an initial interaction between a warhead and a target or may facilitate covalent binding between a warhead and a target. Any multifunctional probe described herein can further include and/or interact with a facilitating functional group. For example, probe 205, shown in FIG. 2A, with a photoreactive warhead 202 of benzophenone, can further include guanidinium ions near (e.g., noncovalently attached to) the benzophenone photoreactive warhead. The guanidinium ions can form a salt bridge between the benzophenone and an anionic oxygen atom (e.g., an oxygen with a negative charge), such as in a protein or lipid. This salt bridge interaction can lead to formation of a covalent bond between the benzophenone and the target.

For example, if a warhead 202, such as a benzophenone warhead, has an affinity for (e.g., preferentially interacting and/or preferentially reacting with) a glycine residue(s) and/or a methionine residue(s), a corresponding warhead preferred warhead binding region 342 may contain one or more glycine residue(s) and/or one or more methionine residue(s). In some examples, a warhead preferred warhead binding region 342 may be glycine and/or methionine rich and may contain a plurality of glycine residues and/or a plurality of methionine residues. For example, a glycine-methionine rich warhead preferred warhead binding region may contain at least two, at least three, at least four, at least five, at least six, at least seven, or at least eight glycine residues and/or methionine residues. The glycine residues and methionine residues may be adjacent to one another or may be non-adjacent in which other, non-methionine and non-glycine residues are interspersed between the glycine and/or methionine residues. From one other, non-methionine and non-glycine residues residue to fifteen other, non-methionine and non-glycine residues may be interspersed between a plurality of glycine and/or methionine residues. For example, if a warhead 202, such as benzophenone or diazirine, has an affinity for (e.g., preferentially interacting and/or preferentially reacting with) a C—H rich region, a corresponding warhead preferred warhead binding region 342 may contain one or more C—H region(s). In some examples, a warhead preferred warhead binding region 342 may be a C—H rich region and may contain a plurality of C—H regions. For example, a C—H region rich warhead preferred warhead binding region may contain at least two, at least three, at least four, at least five, at least six, at least seven, or at least eight C—H regions (such as having one or more arginine, isoleucine, leucine, lysine, etc.) The C—H regions may be adjacent to one another or may be non-adjacent in which other, non-C—H rich regions are interspersed between C—H regions. From one other, non-C—H region to fifteen other non-C—H rich regions (e.g., amino acid residues), may be interspersed between a plurality of C—H rich residues. In some examples, a warhead preferred warhead binding region 342 may be include one or more phenylalanines (e.g., 1, 2, 3, 4, 5, which may be adjacent or interspersed between other amino acids or other chemical structures). A warhead preferred warhead binding region 342 containing one or more phenylalanines may be a preferred binding partner for diazirine.

Examples of tagged warhead preferred warhead binding molecules useful with light-activated warheads such as benzophenone, phenyl azide, and diazirine, are listed in Table 1, where [Cya] is cysteic acid:

| No. | Sequence (N to C) |
|---|---|
| B1 (SEQ ID NO: 16) | Biotin-FMGMGGGGS[Cya]F |
| B2 (SEQ ID NO: 17) | Biotin-GFMMGGGGS[Cya]F |
| B3 (SEQ ID NO: 18) | Biotin-KPKDTLMISR[Cya]F |
| B4 (SEQ ID NO: 19) | Desthiobiotin-FMGMGGGGS |
| | [Cya]F |
| B5 (SEQ ID NO: 20) | Biotin-GMGGE |
| B6 (SEQ ID NO: 21) | Biotin-MGMGE |

A warhead preferred warhead binding molecule may be added to a biomolecular target sample at a concentration of at least 0.01 M, at least 0.1 M, at least 0.2 M, at least 0.3 M, at least 0.5 M and/or less than 1 M, less than 0.9 M, less than 0.8 M, less than 0.7 M, less than 0.6 M, less than 0.5 M, less than 0.1 M, or less than 0.05 M. A warhead preferred warhead binding molecule may be no more than 2 kDa, no more than 1.5 kDa, no more than 1.0 kDa, no more than 0.5 kDa, no more than 0.1 kD and/or at least 0.1 kDa, at least 0.5 kDa, at least 1.0 kDa. For example, a warhead preferred warhead binding molecule may be about 1 kDa. These concentrations may have acceptable specificity (e.g., in light-activated vs non-light-activated regions).

Multifunctional Probe for Light-Induced Capturing

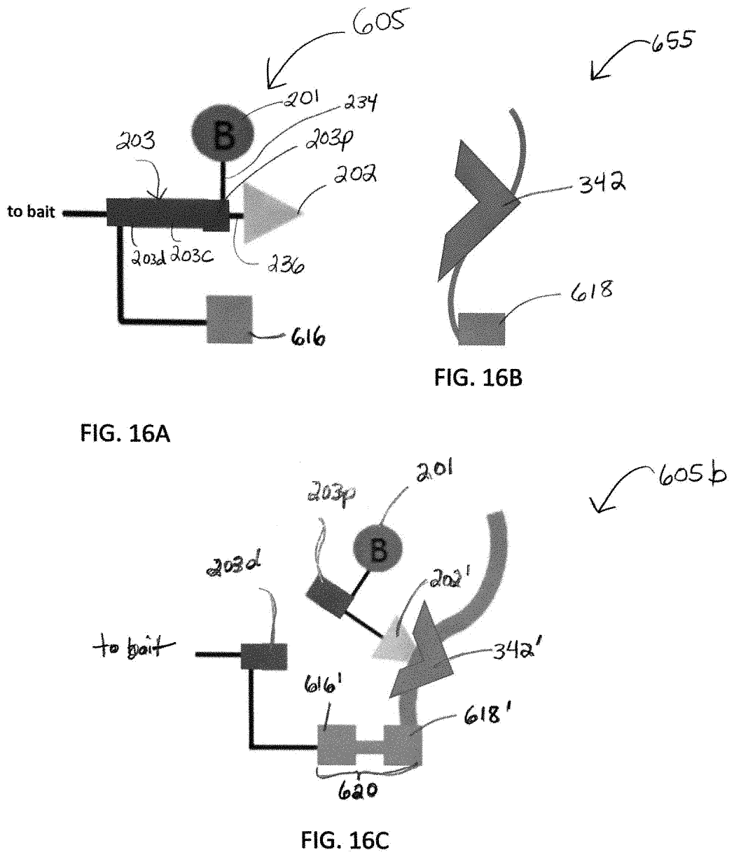

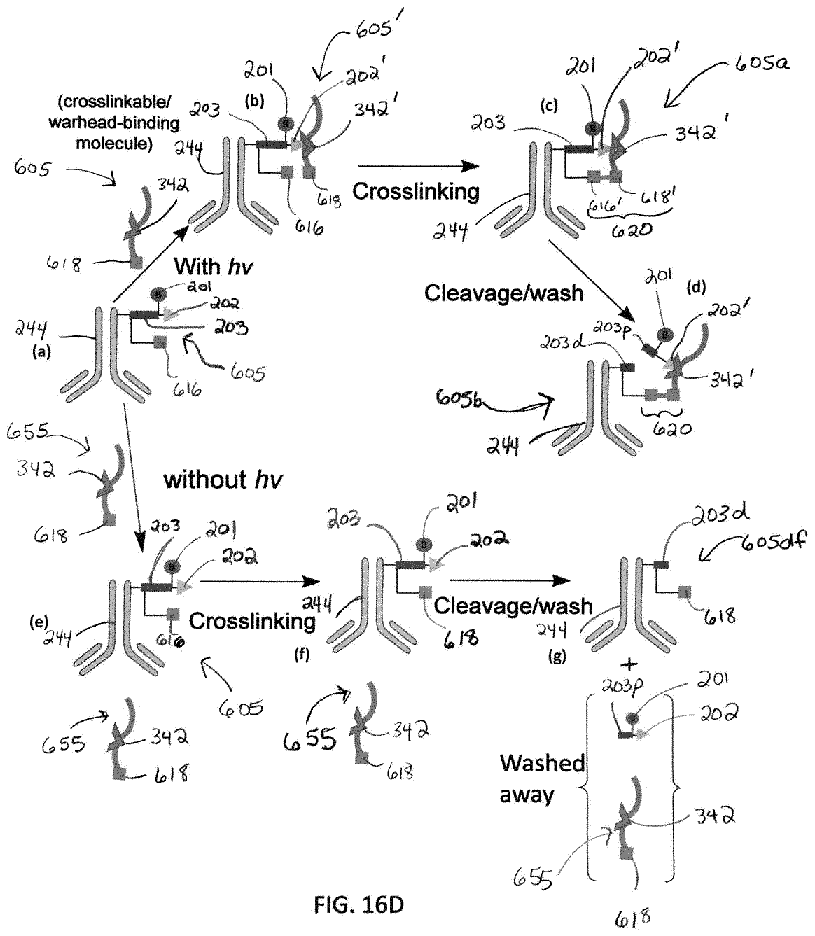

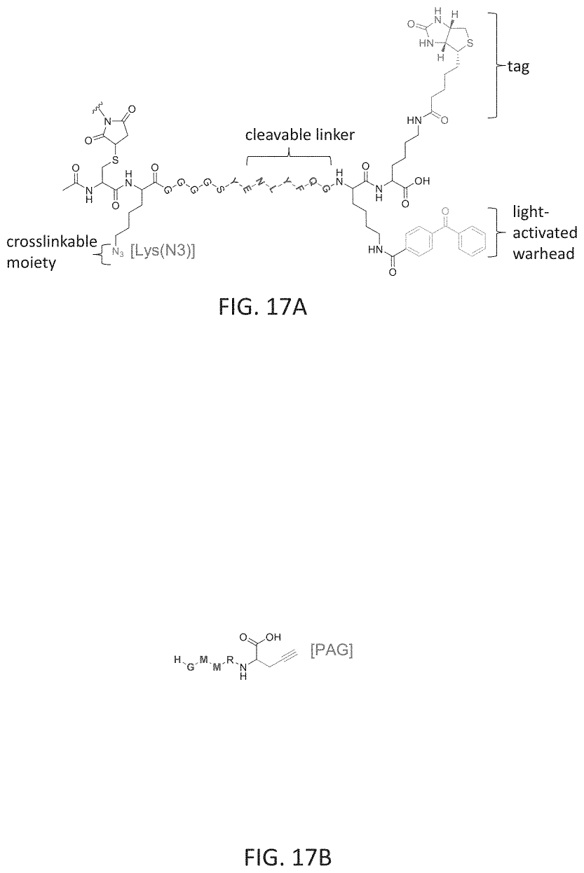

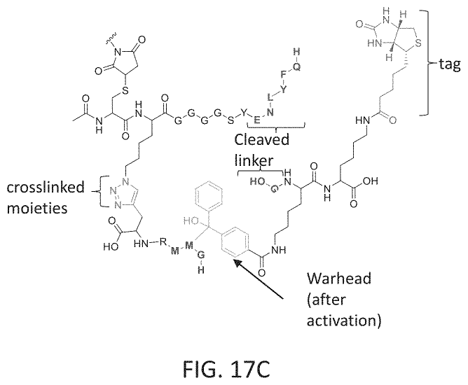

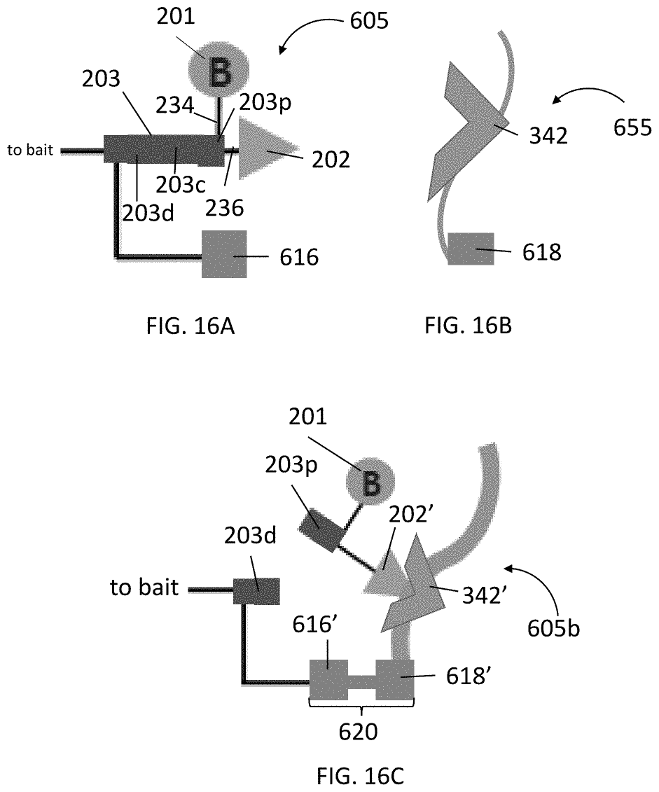

Also described herein are other compositions and methods useful for tagging target molecules, such as protein, nucleic acid, carbohydrate, or lipid biomolecules in an organelle or cell as shown in FIG. 1 and FIG. 2C, or in a cell or tissue extract. After tagging, target molecules may be detected using a detectable label, such as a biotin label, a fluorescent label, horseradish peroxidase, an immunologically detectable label (e.g., a hemagglutinin (HA) tag, a poly-histidine tag), another light emitting label, or a radioactive label. These photoreactive and cleavable probes include a light-activated warhead and a crosslinkable group and can react with connector molecules that contain a warhead binding partner that can bind to the light-activated warhead on the probe. The connector molecules can also include a crosslinkable moiety that can bind to the crosslinkable group on the probe. Target molecules can be selectively labeled in only a region of interest by selectively illuminating only the region of interest (see FIGS. 16A-17C). During target molecule tagging, the pair of crosslinkable (e.g., clickable) groups selectively bind together to thereby attach the tag to the target molecule. The photoreactive and cleavable probe can have the formula (I):

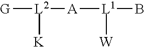

The probe includes a cleavable linker of the probe including: an L1 portion at a proximal region of the cleavable linker; an L2 portion at a distal region of the cleavable linker; and an A portion comprising a cleavage site. The probe can also include W, a light-activated warhead covalently bound to the proximal region of the cleavable linker. The probe can also include B, a tag bound to the proximal region of the cleavable linker. The probe can also include K, a crosslinkable group bound to the distal region of the cleavable linker. The probe can also include G, a bait molecule bound to the distal region of the cleavable linker. FIG. 16A shows a schematic illustration of a photoreactive and cleavable probe, FIG. 16B shows a schematic illustration of a connector molecule configured to react with the photoreactive and cleavable probe illustrated in FIG. 16A, and FIG. 16C shows the probe and connector molecules bound together and cleaved. FIG. 16A illustrates photoreactive and cleavable probe 605 with cleavable linker 203 with multiple attachment sites and multiple moieties attached to cleavable linker 203 by the multiple attachment sites. Cleavable linker 203 includes proximal region 203p (also referred to herein as L¹), distal region 203d (also referred to herein as L²), and cleavage site 203c (also referred to herein as A). Along the length of the probe, distal is closer to the region linked to (or configured to directly link to) the bait and proximal is further away from the region linked to or configured to directly link to) the bait. Cleavage site 203c is between proximal region 203p and distal region 203d (e.g., proximal region 203p is attached to one side of cleavage site 203c and distal region 203d is attached to a different side of cleavage site 203c). FIG. 16A also shows tag 201 (also referred to herein as tag B) bound to a first of the attachment sites at proximal region 203p of cleavable linker 203. Tag 201 is configured to conjugate to a detectable label, as described elsewhere herein. FIG. 16A also shows light-activated warhead 202 (also sometimes referred to herein as a photoreactive warhead) bound to a second of the attachment sites at proximal region 203p of cleavable linker 203. FIG. 16A also shows crosslinkable moiety 616 bound to distal portion 203d of the cleavable linker. Crosslinkable moiety 616 is bound distal to cleavage site 203c of cleavable linker 203. FIG. 16A also shows a bait molecule or a bait molecule attachment site bound to distal portion 203d of cleavable linker 203. The bait molecule or bait molecule attachment site is bound distal to cleavage site 203c of cleavable linker 203.

FIG. 16B schematically illustrates connector molecule 655. In some examples, connector molecule 655 may be especially useful as a double-binding partner to photoreactive and cleavable probe 605. A connector molecule can be functionalized with a warhead binding region and a crosslinkable moiety. Connector molecule 655 includes warhead binding region 342 and crosslinkable moiety 618. A preferential binding partner for the crosslinkable moiety 618 is crosslinkable moiety 616 on probe 605.

FIG. 16C shows probe 605b after probe 605 and connector molecule 655 have been double bound and then cleaved to connect tag 201 and target (not shown in this view; but connected to the bait region). (As described in more detail below with reference to FIG. 16D, this sequence of reactions occurs in a light-activated region and does not occur in a non light-activated region). Warhead 202 has been light-activated and bound to warhead binding region 342 on connector 655, such that warhead 202′ (as modified by light-activation and binding to connector 655) and warhead binding region 342′ (as modified by binding to warhead 202) have been linked together (e.g., covalently bound). Crosslinkable moiety 616 and crosslinkable moiety 618 have also been linked together (e.g., covalently bound) to form linked group 620 with subunits crosslinkable moiety 616′ and crosslinkable moiety 618′ derived from crosslinkable moiety 616 and crosslinkable moiety 618, respectively. Cleavable linker 203 has been cleaved into proximal region 203p and distal region 203d. Proximal region 203p is connected to the warhead and bait. Distal region 203d is no longer bound to proximal region 203p through the cleavable site of the cleavable linker. When the probe 605b is attached to a bait molecule which in turn is attached to a molecule of interest to which the bait molecule binds, the molecule of interest is now tagged with the tag 201 in a region of interest that has been light-activated. FIG. 16D schematically illustrates how molecules in regions that have not been light-activated are not tagged, even if molecules recognized by the bait molecule are present. FIG. 16D schematically illustrates how the probe 605 (see FIG. 16A) and connector molecule 655 are double bound when activated by light to attach tag 201 to bait molecule (antibody 244 in this example, though other bait molecules can instead be used) and through bait molecule (antibody 244 in this example) to a target biomolecule (top series panels (a), (b), (c), and (d). In panel (b), probe 605′ can be formed as light-activated warhead 202′ and warhead binding 342′ are covalently bound together after light activation to join connector 342 to probe 605. In panel (c), probe 605a is formed as the crosslinkable moiety 616 and crosslinkable moiety 618 are covalently bound together crosslinkable moiety 616′ and crosslinkable moiety 618′, respectively.

In the absence of light (bottom series panels (a), (e), (f), and (g)), light-activated warhead 202 is not activated (panel (e)) and warhead 202 and warhead binding partner 342 do not bind (panel (f)) and probe 605 and connector 655 do not bind. The crosslinkable moiety 616 and crosslinkable moiety 618 are not brought into proximity and do not link together (or even if they do, the warhead 202 and warhead binding partner 342 still cannot bind. When cleavable linker 203 is cleaved, the fragment of the probe 605 frag containing tag 201 is not attached to connector 655 and it can be removed (washed away; panel (g)). In this series, bait molecule (antibody 244) (even if attached to a biomolecule) does not get tagged. In these non-lighted regions, connector molecule 655 does not get bound to probe 605 and connector molecule 655 can be removed (washed away). FIGS. 17A-17C show examples of compositions provided herein that can be used with these methods. 17A shows an example of a light-activated probe as schematically illustrated in FIG. 16A with a cleavable linker, detectable tag, and light-activated warhead. FIG. 17A also shows a crosslinkable moiety on the light-activated probe. FIG. 17B shows a connector molecule as schematically illustrated in FIG. 16B, with a warhead binding region and a crosslinkable moeity. FIG. 17C show the photoreactive and cleavable probe shown in FIG. 17A and the connector molecule shown in FIG. 17B doubly connected and then cleaved at the cleavable linker site. Other examples of crosslinkable/warhead-binding molecules include those listed in Table 2:

TABLE 2

| No. | Sequence (N to C) |
|---|---|
| C1 (SEQ ID NO: 24) | GMMR[PAG] |
| C2 (SEQ ID NO: 25) | Ac-GMMR[Lys-(PEG)-alkyne] |
| C3* (SEQ ID NO: 26) | Ac-GMMR[Lys(N3)] |
| C4* (SEQ ID NO: 27) | Ac-DTLMISR[Lys(N3)] |

In Table 2, [PAG] refers to Propargyl Glycine. For C3 and C4, additional linkers such as bis-propargyl-PEG can be used to conduct post-illumination crosslinking. Lys-(PEG)-alkyne] refers to:

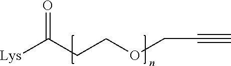

In some variations, probe 605 includes bait bound to the distal region of the cleavable linker and in some variations, probe 605 includes a bait attachment region bound to the distal region of the cleavable linker and configured to link to a bait molecule. A probe can be linked to a bait molecule prior to carrying out a tagging reaction, such as the one shown in FIG. 16A-FIG. 16D. Bait can be e.g., an antibody, a secondary antibody, a CLIP-tag (e.g., a fluorophore conjugated to a cytosine leaving group via a benzyl linker), a HaloTag (a protein tag such as a modified haloalkane dehalogenase designed to covalently bind to synthetic ligands (HaloTag ligands), protein A, protein G, protein L, an RNA molecule, a small molecule, or a SNAP-tag (e.g., a ˜20 kDa mutant of the DNA repair protein O6-alkylguanine-DNA alkyltransferase that reacts specifically and rapidly with benzylguanine (BG) derivatives) or other bait molecules. Crosslinkable moiety 616 and crosslinkable moiety 618 can be specifically interacting binding partners, such as “click chemistry” groups or moieties (e.g., a 2-cyano-6-aminobenzothiazole (CBT) moiety, a strained alkene, an alkyne based moiety, a strained alkyne moiety, a terminal alkyne moiety, an azide based moiety, a tetrazine moietry, a trans-cyclooctene moiety (TCO), etc.). In some variations, a cleavable linker (e.g., cleavable linker 203) includes a cleavable linker bond other than a disulfide bond. A cleavable linker can have a bioorthogonal cleavage site and have a site that is not present in cells, cell types, or tissues of interest. A cleavable linker can have a specific bioorthogonal cleavage site of interest, such as a mammalian bioorthogonal site (e.g., have a linker cleavage site that is not present in mammalian tissue), a eukaryotic bioorthogonal site (e.g., have a linker cleavage site that is not present in mammalian tissue), a prokaryotic bioorthogonal site (e.g., have a linker cleavage site that is not present in prokaryotes), etc. A cleavable linker can include an azobenzene derivative, a boronic acid ester, a Dde derivative, a DNA oligomer, and/or a specifically cleavable peptide. In some examples, a cleavable linker includes a bioorthogonal protease-cleavable peptide chain. A cleavable linker can include a human rhinovirus 3C (HRV 3C) protease cleavage site (and cleaved by the addition of human rhinovirus 3C (HRV 3C) protease), a tobacco etch virus (TEV) protease cleavage site (and cleavable by tobacco etch virus (TEV) protease), and/or a thrombin cleavage site (and cleavable by thrombin). Crosslinkable moiety 616 and crosslinkable moiety 618 may include bioorthogonal moieties that are non-naturally occurring and do not readily bind to naturally occurring molecules in a target sample. The crosslinkable moiety 616 and crosslinkable moiety 618 preferentially bind to each other. (They may be configured to not bind to another region on the probe or to another region on the connector or to any endogenously occurring molecules in a sample or any suspected endogenously occurring molecules in a sample). They may be configured to not bind to any naturally occurring mammalian molecules, any naturally occurring eukaryotic molecules, any naturally occurring prokaryotic molecules, etc. A tag of a probe (such as probe 605) can include a biotin derivative, a click chemistry tag, a CLIP-tag, a digoxigenin tag, a HaloTag, a peptide tag, or a SNAP-tag or any other tags, including any others disclosed elsewhere herein. A light-activated warhead of a probe (such as probe 605) can include an aryl azide, a benzophenone, a diazirine, such as:

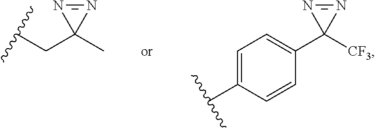

a nucleobase-specific 3-cyanovinylcarbazole nucleoside (CNVK), such as

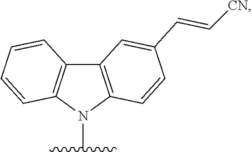 [SMILES: *n1c2ccccc2c2cc(/C=C/C#N)ccc21]

a nucleobase-specific psoralen, such as

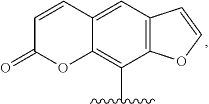 [SMILES: *c1c2occc2cc2ccc(=O)oc12]

a phenoxyl radical, and/or a phenoxy radical trapper such as

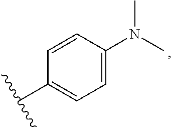 [SMILES: *c1ccc(N(C)C)cc1]

In some variations, in a photoreactive and cleavable probe, such as probe 605, with any of the variations described herein, such as the moiety of formula (I-1), a tag (B) and warhead (W) are bound to a proximal region of the cleavable linker through the moiety of formula (I-1):

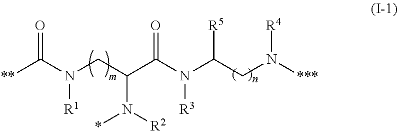

wherein m and n each independently are 1, 2, 3, 4, 5, or 6; the symbol * is a first attachment site for a proximal end of the “A” portion; the symbol ** is a second attachment site for one of the tag and the light-activated warhead; the symbol *** is a third attachment site for another one of the tag and the light-activated warhead (e.g., whichever is not bound to the second attachment site); R¹, le, R³ and R⁴ each independently are hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, or a nitrogen protecting group; and R⁵ is —COORa, wherein Ra is hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, or an oxygen protecting group. In some variations, in a photoreactive and cleavable probe, such as probe 605, with any of the variations described herein, wherein the L² portion includes a moiety of formula (I-2), and the G and the K are bound to the distal region of the cleavable linker through the moiety of formula (I-2):

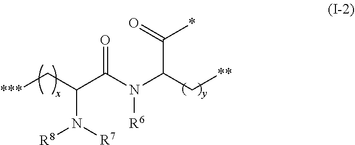

wherein x and y each independently are 1, 2, 3, 4, 5, or 6; the symbol * is a first attachment site for a distal end of the “A” portion; the symbol ** is a second attachment site for one of the crosslinkable group and the bait molecule or the bait attachment site; the symbol *** is a third attachment site for another one of the crosslinkable group and the bait molecule or the bait attachment site; and R⁶, le and R⁸ each independently are hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, or a nitrogen protecting group.

As indicated above, a connector molecule can include a warhead binding region, wherein the warhead binding region preferentially binds to a light-activated warhead; and a crosslinkable moiety, wherein the crosslinkable moiety preferentially binds to a crosslinkable moiety binding partner. In some examples, the warhead binding region of a connector molecule preferentially binds an aryl azide light-activated warhead, a benzophenone light-activated warhead, or a diazirine light-activated warhead. In some examples, warhead binding region of a connector molecule includes an aliphatic C—H region (such as any of those indicate above). In some examples, a crosslinkable moiety of a connector molecule includes a click chemistry-based moiety.

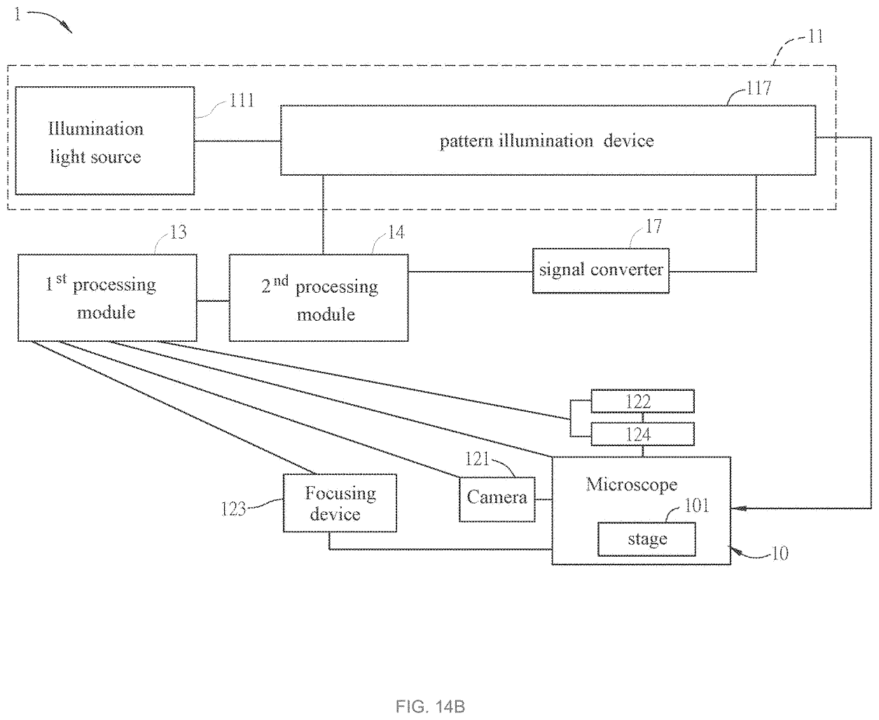

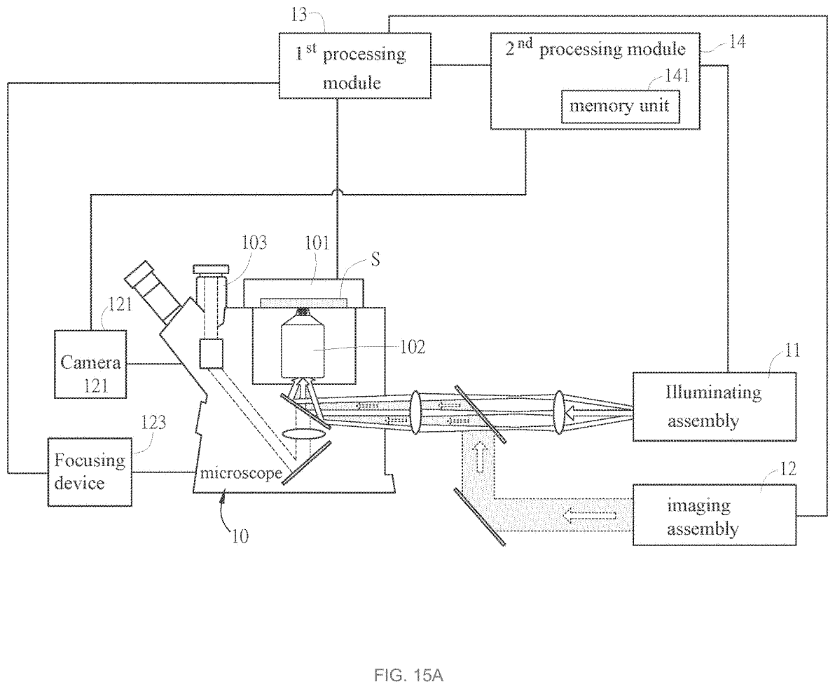

This disclosure provides an embodiment which is also a microscope-based system for image-guided microscopic illumination. Please refer to FIGS. 14A and 14B. The microscope-based system of this embodiment comprises a microscope 10, an imaging assembly 12, an illuminating assembly 11, and a processing module 13a. The microscope 10 comprises an objective 102 and a stage 101. The stage 101 is configured to be loaded with a sample S. The imaging assembly 12 may comprise a (controllable) camera 121, an imaging light source 122, a focusing device 123, and a first shutter 124. Please further refer to FIG. 14B, the illuminating assembly 11 may comprise an illumination light source 111 and a pattern illumination device 117. The pattern illumination device 117 may include a second shutter 112, a lens module 113 (such as the relay lens 113a and 113b, a quarter wave plate 113c), at least a pair of scanning mirrors 115 and a scan lens 116. Alternatively, digital micromirror device (DMD) or spatial light modulator (SLM) can be used as the pattern illumination device 117.

In this embodiment, the processing module 13a is coupled to the microscope 10, the imaging assembly 12, and the illuminating assembly 11. The processing module 13a can be a computer, a workstation, or a CPU of a computer, which is capable of executing a program designed for operating this system.

The processing module 13a controls the imaging assembly 12 such that the camera 121 acquires at least one image of the sample S of a first field of view, and the image or images are transmitted to the processing module 13a and processed by the processing module 13a automatically in real-time based on a predefined criterion, so as to determine an interested region in the image S and so as to obtain a coordinate information regarding to the interested region. Later, the processing module 13a may control the pattern illumination device 117 of the illuminating assembly 11 to illuminate the interested region of the sample S according to the received coordinate information regarding to the interested region. Also, after the interested region is fully illuminated, the processing module 13a controls the stage 101 of the microscope 10 to move to a second field of view which is subsequent to the first field of view.

In this embodiment, the imaging light source 122 provides an imaging light through an imaging light path to illuminate the sample S during imaging the sample. The first shutter 124, along the imaging light path, is disposed between the image light source 122 and the microscope 10. The controllable camera 121 is disposed on the microscope 10 or on the imaging light path.

Also, the illuminating light source 111 provides an illuminating light through an illuminating light path to illuminate the sample S. The pattern illumination device 117, along the illuminating light path, is disposed between the illumination light source 111 and the microscope 10.

This disclosure provides another embodiment which is also a microscope-based system for image-guided microscopic illumination. This system includes an additional processing module to improve illumination performance and will be describe in detail. Please refer to FIGS. 14A and 14B. FIG. 14A represents a schematic diagram of an imaging-guided system according to one embodiment of the present disclosure, and FIG. 14B depicts the optical path of the image-guided system of FIG. 14A.

As shown in FIGS. 14A and 14B, the microscope-based system 1 for image-guided microscopic illumination comprises a microscope 10, an illuminating assembly 11, an imaging assembly 12, a first processing module 13 and a second processing module 14. The microscope-based system 1 is designed to take a microscope image or images of a sample and use this image or these images to determine and shine an illumination pattern on the sample, finishing all steps for one image rapidly (e.g. within 300 ms), and within a short time (e.g. 10 hours) for the entire illumination process for a proteomic study.

The microscope 10 comprises a stage 101, an objective 102 and a subjective 103. The stage is configured to be loaded with a sample S. The stage 101 of the microscope 10 can be a high-precision microscope stage.

The imaging assembly 12 may comprise a camera 121, an imaging light source 122, a focusing device 123, and a first shutter 124. The camera 121 is mounted on the microscope 10. In detail, the camera 121 is coupled to the microscope 10 through the subjective 103 of the microscope 10. The focusing device is coupled to the camera 121 and controlled to facilitate an autofocusing process during imaging of the sample S. The imaging light source 122, which provides an imaging light (as shown in the shaded area in FIG. 14A from imaging assembly 12 to the objective 102) through an imaging light path (as shown with the route indicated by the open arrows in the shaded area depicting the imaging light in FIG. 14A) to illuminate the sample S. The first shutter 124, along the imaging light path, is disposed between the image light source 122 and the microscope 10. The imaging light source 122 can be a tungsten-halogen lamp, an arc lamp, a metal halide lamp, a LED light, a laser, or multiple of them. The shuttering time of the first shutter may vary with the type of the imaging light source 121. Using an LED light source as an example, the shuttering time of the first shutter 124 is 20 microseconds.

If one would like to perform two color imaging, the shutter of the first color light is turned off and the shutter of the second color light is turned on by the first processing module 13. This may take another 40 microseconds. The camera 121 then takes another image with an exposure time of another 20 millisecond. The first processing module 13 then turns off the shutter of the second color light.

In this embodiment, please further refer to FIG. 14B, the illuminating assembly 11 comprises an illuminating light source 111, and a pattern illumination device 117 including a second shutter 112, a lens module 113 (such as the relay lens 113a and 113b, a quarter wave plate 113c), at least a pair of scanning mirrors 115 and a scan lens 116. Alternatively, DMD or SLM can be used as the pattern illumination device 117. The illuminating light source 111 provides an illuminating light (as shown in the open arrows from the illuminating assembly 11 to the objective 102 in FIG. 14A) through an illuminating light path to illuminate the sample S. The second shutter 112, along the illuminating light path, is disposed between the illuminating light source 111 and the microscope 10. The pair of scanning mirrors 115, along the illuminating light path, is disposed between the second shutter 112 and the microscope 10. The camera 121 may be a high-end scientific camera such as an sCMOS or an EMCCD camera with a high quantum efficiency, so that a short exposure time is possible. To get enough photons for image processing, the exposure time is, for example, 20 milliseconds.

The first processing module 13 is coupled to the microscope 10 and the imaging assembly 12. In detail, the first processing module 13 is coupled and therefore controls the camera 121, the imaging light source 122, the first shutter, the focusing device 123, and the stage 101 of the microscope 10, for imaging, focus maintenance, and changes of fields of view. The first processing module 13 can be a computer, a workstation, or a CPU of a computer, which is capable of executing a program designed for operating this system. The first processing module 13 then triggers the camera 121 to take the image of the sample S of a certain field of view (FOV). In addition, the camera 121 can be connected to the first processing module 13 through an USB port or a Camera Link thereon. The controlling and the image-processing procedures of this system will be discussed more detailed in the following paragraphs.

In this embodiment, the second processing module 14 is coupled to the illuminating assembly 11 and the first processing module 13. In detail, the second processing module 14 is coupled to and therefore controls the pattern illumination device 117, including the second shutter 112, and the pair of scanning mirrors, for illuminating the targeted points in the interested region determined by the first processing module 13. The second processing module may be a FPGA, an ASIC board, another CPU, or another computer. The controlling and the image-processing procedures of this system will be discussed more detailed in the following paragraphs.

In brief, the microscope-based system 1 is operated as below. The first processing module 13 controls the imaging assembly 12 such that the camera 121 acquires at least one image of the sample S of a first field of view. The image or images are then transmitted to the first processing module 13 and processed by the first processing module 13 automatically in real-time based on a predefined criterion, so as to determine an interested region in the image and so as to obtain a coordinate information regarding to the interested region. The image processing algorithm is developed independently beforehand using image processing techniques such as thresholding, erosion, filtering, or artificial intelligence trained semantic segmentation methods. Later, the coordinate information regarding to the interested region is transmitted to the second processing module 14. The second processing module 14 controls the illuminating assembly 12 to illuminate the interested region (or, namely, irradiating those targeted points in the interested region) of the sample S according to the received coordinate information regarding to the interested region. In addition, after the interested region is fully illuminated (or all the targeted points in the interested region are irradiated), the first processing module 13 controls the stage 101 of the microscope 10 to move to the next (i.e. the second) field of view which is subsequent to the first field of view. After moving to the subsequent field of view, the method further repeats imaging-image processing-illumination steps, until interested regions of all designated fields of view are illuminated.

Moreover, this disclosure also provides another embodiment which is a microscope-based method for image-guided microscopic illumination. The microscope-based method uses the microscope-based system described above and comprises the following steps (a) to (e): (a) triggering the camera 121 of the imaging assembly 12 by the first processing module 13 to acquire at least one image of the sample S of a first field of view, and the sample S is loaded on the stage 101 of the microscope 10; (b) automatically transmitting the image or images of the sample S to the first processing module 13; (c) based on a predefined criterion, performing image processing of the sample S automatically in real-time by the first processing module 13 to determine an interested region in the image and obtain a coordinate information regarding to the interested region; (d) automatically transmitting the coordinate information regarding to the interested region to the second processing module 14; (e) controlling an illumination assembly 11 by the second processing module 14 according to the received coordinate information to illuminate the interested region in the sample S. Besides, in this embodiment, after the interested region is fully illuminated, the method may further comprise a step of: controlling the stage 101 of the microscope 10 by the first processing module 13 to move to the next (i.e. the second) field of view which is subsequent to the first field of view.

The microscope-based system 1 used herein are substantially the same as that described above, and the details of the composition and variations of the compositing elements are omitted here.

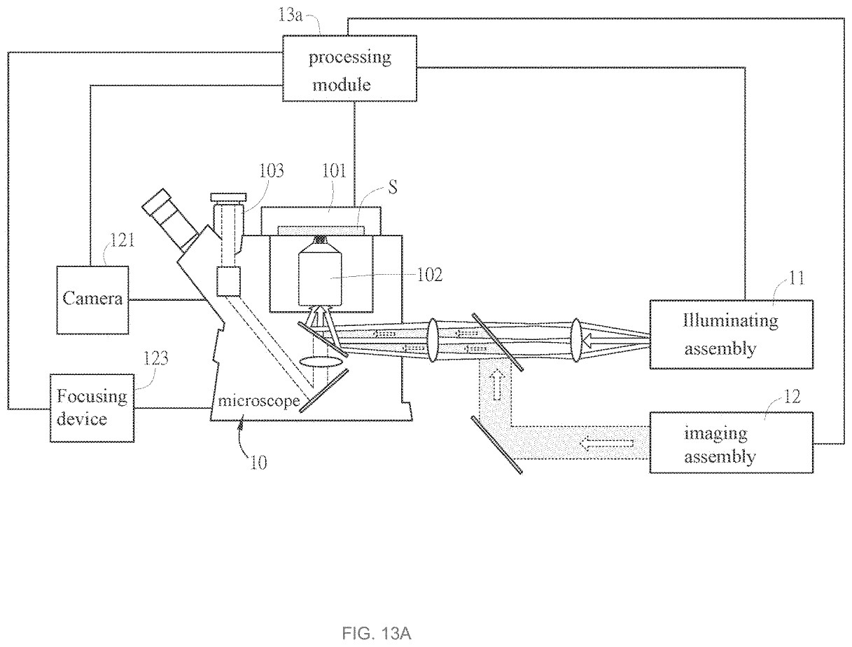

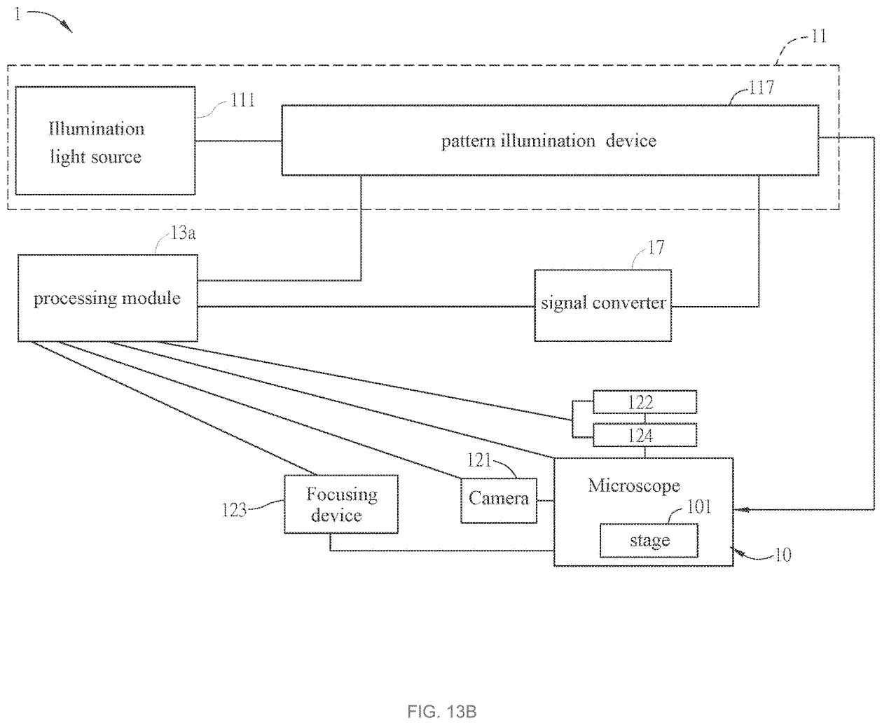

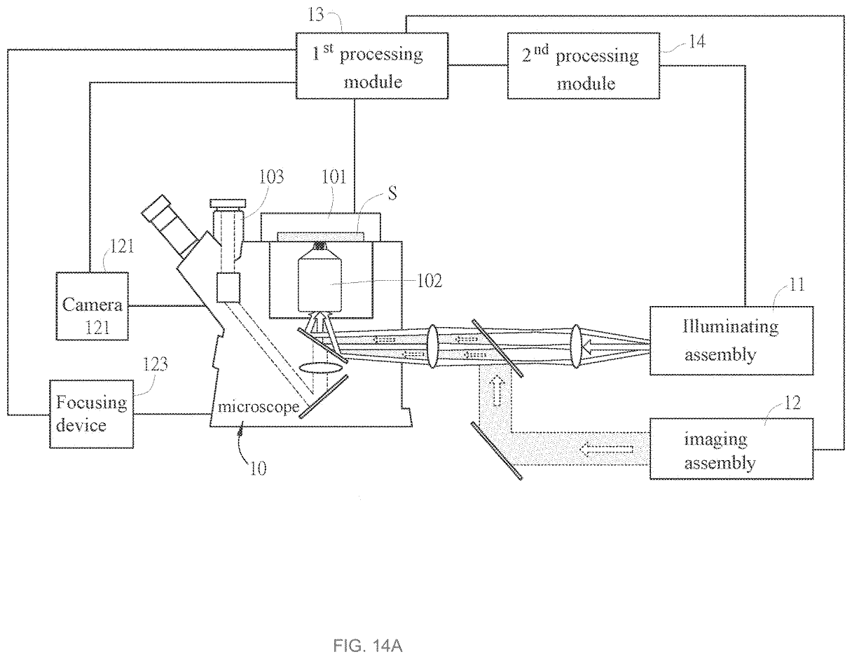

Moreover, as shown in FIGS. 13A and 13B, the light path of the illumination starts from the illumination light source 111. The second shutter 112 is needed for this illumination light source 111. To reach a high switching speed for the point illumination, a mechanical shutter may not be fast enough. One may use an acousto-optic modulator (AOM) or an electro optic modulator (EOM) to achieve the high speed. For example, an AOM can reach 25-nanosecond rise/fall time, enough for the method and system in this embodiment. After the second shutter 112, the beam size may be adjusted by a pair of relay lenses 113a and 113b. After the relay lenses 113a and 113b, the quarter wave plate 113c may facilitate to create circular polarization. The light then reaches the pairs of scanning mirrors (i.e. XY-scanning mirrors) 115 to direct the illumination light to the desired point one at a time. The light then passes a scan lens 116 and a tube lens (included in a microscope, not shown here) and the objective 102 of the microscope 10 to illuminate the targeted point of the sample S. An objective 102 with a high numerical aperture (NA) may be needed to have enough light intensity for photochemical reactions or photoconversion.

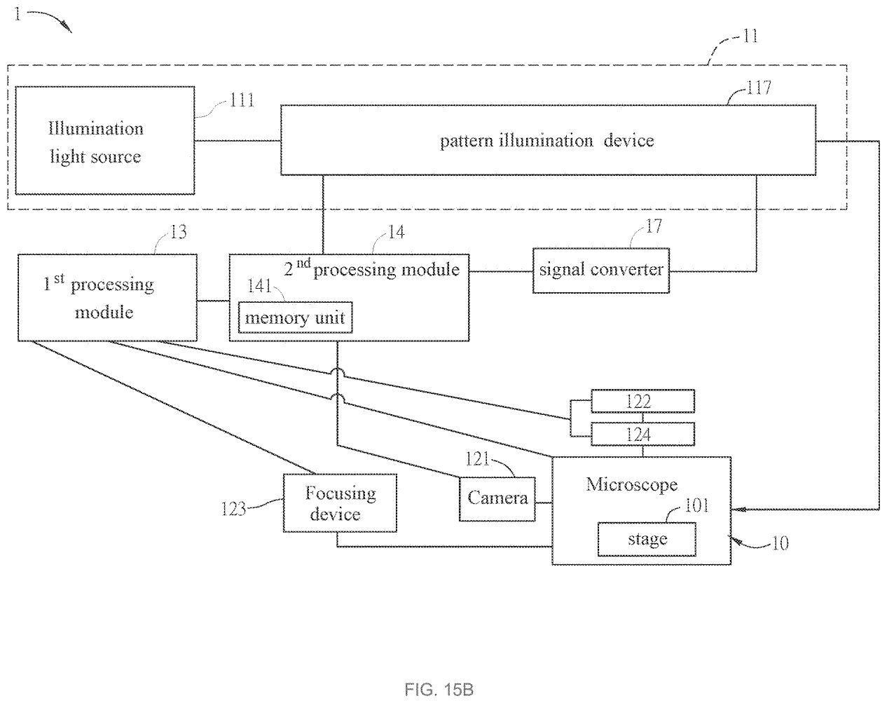

Also, this disclosure also provides another embodiment which is another microscope-based system for image-guided microscopic illumination. The microscope-based system for image-guided microscopic illumination is substantially the same as that is described above. Please refer to FIGS. 15A and 15B. In this embodiment, the microscope-based system 1 comprises a microscope 10, an illuminating assembly 11, an imaging assembly 12, a first processing module 13 and a second processing module 14. The microscope 10 comprises a stage 101, an objective 102 and a subjective 103, and the stage 10 is configured to be loaded with a sample S. Please further refer to both FIG. 15B, the illuminating assembly 11 comprises an illuminating light source 111, and a pattern illumination device 117 including a second shutter 112, at least one relay lens (such as the relay lens 113a and 113b), a quarter wave plate 113c, at least a pair of scanning mirrors 115 and a scan lens 116. Alternatively, DMD or SLM can also be used as the pattern illumination device 117. The imaging assembly 12 may comprise a camera 121, an imaging light source 122, a focusing device 123, and a first shutter 124. The camera 121 is mounted on the microscope 10.

The major difference between the systems described in the previous embodiment and here is that the first processing module 13 here is coupled to the stage 101 of the microscope 10 and the imaging light source 122 and the first shutter 124 of the imaging assembly 12. However, the second processing module 14 here comprises a memory unit 141 and is coupled to the camera 121, the illuminating assembly 11, and the first processing module 13. In other words, in this embodiment, the camera 121 is controlled by the second processing module 14 instead of the first processing module (i.e. the computer) 13. The camera 121 can be connected to the second processing module 14 through a Camera Link if a high speed of image data transfer and processing is required. The memory unit 141 can be a random access memory (RAM), flash ROM, or a hard drive, and the random access memory may be a dynamic random access memory (DRAM), a static random access Memory (SRAM), or a zero-capacitor random access memory (Z-RAM).

Hence, in the system 1 embodied here, it is operated as follows. The first processing module 13 controls the imaging assembly 12 and the second processing module controls 14 the camera 121 such that the camera 121 acquires at least one image of the sample S of a first field of view. The image or images are then automatically transmitted to the memory unit 141 to the second processing module 14. Image processing is then performed by the second processing module 14 automatically in real-time based on a predefined criterion, so as to determine an interested region in the image and so as to obtain a coordinate information regarding to the interested region. Later, the second processing module 14 controls the illuminating assembly 11 to illuminate the interested region of the sample S according to the received coordinate information regarding to the interested region.

Because composition, variation or connection relationship to other elements of each detail elements of the microscope-based system 1 can refer to the previous embodiments, they are not repeated here.

Also, this disclosure also provides still another embodiment which is another microscope-based method for image-guided microscopic illumination. The microscope-based method for image-guided microscopic illumination is substantially the same as that is described above. Please also refer to FIGS. 15A and 15B, the microscope-based method for image-guided microscopic illumination comprises the following steps through (a) to (d): (a) controlling the imaging assembly 12 by the first processing module 13 and triggering the camera 121 of the imaging assembly 12 by the second processing module 14 to acquire at least one image of the sample S of a first field of view, and the sample S is loaded on the stage 101 of the microscope 10; (b) automatically transmitting the image or images of the sample S to the memory unit 141 of the second processing module 14; (c) based on a predefined criterion, performing image processing of the sample S automatically in real-time by the second processing module 14 to determine an interested region in the image and to obtain a coordinate information regarding to the interested region; and (d) controlling the illuminating assembly 11 by the second processing module 14 to illuminate the interested region in the sample S according to the received coordinate information.

The wavelength of light for performing warhead activation or photoselective tagging and labeling ranges in some embodiments from about 200 nm to about 800 nm, e.g., from about 200 nm to about 250 nm, from about 250 nm to about 300 nm, from about 300 nm to about 350 nm, from about 350 nm to about 400 nm, from about 400 nm to about 450 nm, from about 450 nm to about 500 nm, from about 500 nm to about 550 nm, from about 550 nm to about 600 nm, from about 600 nm to about 650 nm, from about 650 nm to about 700 nm, from about 700 nm to about 750 nm, or from about 750 nm to about 800 nm. In some embodiments, the wavelength of light for performing photoselective tagging and labeling is short-wavelength UV light (e.g., 254 nm; 265-275 nm); long-UV light (e.g., 365 nm; 300-460 nm). The wavelength of light for performing warhead activation or photoselective tagging and labeling ranges in some embodiments from about 800 nm to about 2000 nm, e.g., from about 800 nm to about 900 nm, from about 900 nm to about 1000 nm, from about 1000 nm to about 1100 nm, from about 1100 nm to about 1200 nm, from about 1200 nm to about 1300 nm, from about 1300 nm to about 1400 nm, from about 1400 nm to about 1500 nm, from about 1500 nm to about 1600 nm, from about 1600 nm to about 1700 nm, from about 1700 nm to about 1800 nm, from about 1800 nm to about 1900 nm, or from about 1900 nm to about 2000 nm. In some embodiments, the wavelength of light for performing photoselective tagging and labeling is short-wavelength UV light (e.g., 254 nm; 265-275 nm); long-UV light (e.g., 365 nm; 300-460 nm). The wavelengths used for photoactivation of the warhead is different from the wavelengths used for imaging. In some embodiments, photoreactive warhead activation utilizes optical radiation (light) at from around 300-450 nm, 550 nm for single photon activation or >720 nm for multiphoton activation. The particular wavelength depends on the particular warhead. Cleavage can be driven by an enzyme or chemicals (such as sodium dithionite for cleaving azobenzene).

In some embodiments, a multivalent core (e.g., a core moiety) of a probe can be from around 70 Da to about 500 Da. A multivalent core can include or can be a single amino acid or a single nucleotide. In some embodiments, a core can be less than 1 nm in maximal width.

Methods

Also disclosed herein are methods of photoselectively tagging and labeling biomolecules and analytical methods. The methods may be used to tag and/or label carbohydrates, lipids, nucleic acids, proteins, either alone or in combination. The methods may include the step of treating a biological sample with a bait molecule and a photoreactive and mildly cleavable probe and binding the bait molecule to a prey in the biological sample. In some embodiments, the probe includes a light-activated warhead and a tag and is bound to the bait molecule through a cleavable linker. Some embodiments include the step of illuminating the biological sample with an imaging lighting source of an image-guided microscope system. Some embodiments include the step of imaging the illuminated sample with a controllable camera. Some embodiments include the step of acquiring with the camera at least one image of subcellular morphology of the sample in a first field of view. Some embodiments include the step of processing the at least one image and determining a region of interest in the sample based on the processed image. Some embodiments include the step of obtaining coordinate information of the region of interest.

Some embodiments include the step of selectively illuminating with a crosslinking light the region of interest based on the obtained coordinate information to thereby doubly crosslink the probe and the bait. Some embodiments include the step of further comprising using the tag to generate a detectable label and labeling proteins proximal the prey with the detectable label. Some embodiments include the step of wherein the detectable label comprises a tyramine label. Some embodiments include the step of, wherein the biological sample comprises a plurality of cells. Some embodiments include the step of wherein the biological sample comprises a plurality of living cells. Some embodiments include the step of wherein the biological sample comprises cell extracts. Some embodiments include the step of wherein selectively illuminating comprises illuminating a region less than 300 nm, less than 200 nm, or less than 100 nm in diameter. Some embodiments include the step of further comprising removing at least the region of interest from the microscope stage.

Some embodiments include the step of further comprising subjecting the sample to mass spectrometry or sequencing analysis. Some embodiments include the wherein the tag comprises a biotin derivative, a click chemistry tag, a HaloTag, a SNAP-tag, a CLIP-tag, digoxigenin, or a peptide tag. Some embodiments include the wherein the click chemistry tag comprises an alkyne-based or azide-based moiety. Some embodiments include the wherein the cleavable linker is an azobenzene derivative, a Dde derivative, a DNA oligomer, a peptide, or a boronic acid ester. Some embodiments include the wherein the bait molecule comprises an antibody, protein A, protein G, protein L, a SNAP-tag, a CLIP-tag or a small molecule. Some embodiments include the wherein the light-activated warhead comprises an aryl azide, a diazirine, or a benzophenone.

Also described herein are photoselective tagging, labeling, and analyzing methods. methods. The methods may include the step of delivering a photoreactive and cleavable probe to a biological sample, wherein the probe comprises a cleavable linker, a light-activated warhead, and a tag and attached to a core of the probe. The methods may include the step of binding a bait molecule to a target biomolecule in the biological sample, wherein the bait molecule is conjugated to the probe. The methods may include the step of illuminating the biological sample from an imaging lighting source of an image-guided microscope system.

The methods may include the step of imaging the illuminated sample with a controllable camera. The methods may include the step of acquiring with the camera at least one image of subcellular morphology of the biological sample in a first field of view. The methods may include the step of processing the at least one image and determining a region of interest in the sample based on the processed image. The methods may include the step of obtaining coordinate information of the region of interest. The methods may include the step of selectively illuminating the region of interest with optical radiation to activate the light-activated warhead and attach the warhead to the target biomolecule or a target biomolecule neighbor such that the probe and target molecule are double-crosslinked. The methods may include the step of cleaving the cleavable linker of the probe. The methods may include the step of removing the cleaved and unbound probe.

Some embodiments include labeling a region less than 300 nm, less than 200 nm, or less than 100 nm in diameter. In some embodiments, the biological sample includes at least one, at least 100, at least 1000 or at least 10,000 live cells.

Some methods include contacting a biological sample having a target biomolecule with a probe as described herein, using optical radiation to spatially selectively photocrosslink the probe with a target biomolecule, cleaving the probe, washing unbound probe or cleaved probe away, labeling the biomolecule/probe complex with a label, and selectively proximity labeling biomolecule neighbor molecules.

Some embodiments provide a method for photoactivated labeling, the method including delivering a photoreactive and cleavable probe as described herein (e.g., probe 605), conjugating abait molecule to a target biomolecule in a sample; delivering a connector molecule (e.g., connector 655) to the sample, wherein the connector molecule includes a warhead binding region and a crosslinkable moiety. Some methods include selectively illuminating a selected region of interest of the sample with optical radiation, to thereby activate the light-activated warhead and attaching, in the selected region of interest of the sample, the light-activated warhead to the warhead binding region. Some methods include crosslinking the crosslinkable group on the probe with the crosslinkable moiety on the connector molecule to thereby crosslink the tag to the bait through the crosslink. Some methods include cleaving the cleavable linker of the probe with a cleaver, such that tag crosslinked to the bait through the crosslink remains attached to the bait, while tag that is not crosslinked to the bait is removed from the bait. Some methods include removing unbound and cleaved probe and connector molecules.

Some embodiments provide a method for photoactivated labeling, the method including delivering a photoreactive and cleavable probe as described herein (e.g., probe 605) as to a sample and conjugating the bait molecule to a target biomolecule in the sample. Some methods include delivering a connector molecule (e.g., connector molecule 655) to the sample, wherein the connector molecule includes a warhead binding region and a crosslinkable moiety. Some methods include the step of selectively illuminating a selected region of interest of the sample with optical radiation, to thereby activate the light-activated warhead and attaching, in the selected region of interest of the sample, the light-activated warhead to the warhead binding region. Some methods include the step of crosslinking the crosslinkable group on the probe with the crosslinkable moiety on the connector molecule to thereby crosslink the tag to the bait through the crosslink. Some methods include the step of cleaving the cleavable linker of the probe with a cleaver, such that tag crosslinked to the bait through the crosslink remains attached to the bait, while tag that is not crosslinked to the bait is removed from the bait. Some methods include the step of removing unbound and cleaved probe and connector molecules.

Some embodiments provide an analytical method including: delivering a photoreactive and cleavable probe to a biological sample, wherein the probe includes a bait molecule, a cleavable linker, a light-activated warhead, a tag, and a crosslinkable group, wherein the light-activated warhead and the tag are attached to a proximal region of the cleavable linker, and the crosslinkable group and bait are attached to a distal region of the cleavable linker. Some methods include the step of conjugating the probe to a target biomolecule in the biological sample to form a conjugated probe-target biomolecule. Some methods include the step of

illuminating the biological sample from an imaging lighting source of an image-guided microscope system. Some methods include the step of imaging the illuminated sample with a controllable camera. Some methods include the step of acquiring with the camera at least one image of subcellular morphology of the biological sample in a first field of view. Some methods include the step of processing the at least one image and determining a region of interest in the sample based on the processed image. Some methods include the step of obtaining coordinate information of the region of interest. Some methods include the step of selectively illuminating the region of interest with optical radiation to activate the light-activated warhead and attach the warhead to a warhead binding region of a connector molecule, wherein the connector molecule further comprises a crosslinkable moiety. Some methods include the step of crosslinking the crosslinkable group on the probe with the crosslinkable moiety on the connector molecule to thereby crosslink the tag to the bait. Some methods include the step of cleaving the cleavable linker of the probe at a cleavage site between the proximal and distal regions of the cleavable linker, such that tag crosslinked to the bait through the crosslink remains attached to the bait, while tag that is not crosslinked to the bait is removed from the bait. Some methods include the step of removing unbound and cleaved probe and connector molecules.

In some of these methods, cleaving the cleavable linker includes performing a bioorthogonal cleavage reaction. In some of these methods, the cleavable linker includes a cleavable linker bond and the step of cleaving the cleavable linker includes cleaving a bond other than a disulfide bond. Some of these methods include conjugating a detectable label with the tag of the probe and detectably proximity labeling neighbors proximal the target biomolecule by detectable label activity. In some of these methods, selectively illuminating includes illuminating a region for 25 us/pixel to 400 us/pixel, for 50 us/pixel to 300 us/pixel, or for 75 us/pixel to 200 us/pixel. In some of these methods, selectively illuminating includes illuminating with a power intensity of from 100 mW to 300 mW. In some of these methods, selectively illuminating comprises illuminating a zone defined by point spread function. In some of these methods, detectably proximity labeling includes photoselective proximity labeling a region less than 300 nm, less than 200 nm, or less than 100 nm in diameter. In some of these methods, the detectable label includes a catalytic label. In some of these methods, the biological sample includes at least one, at least 100, at least 1000 or at least 10,000 live or fixed cells. In some of these methods, the biological sample includes fixed cells, fixed tissues, cell extracts, or tissue extracts. In some of these methods, the biological sample is disposed on a microscope stage, the method further includes removing at least a portion of the biological sample region of interest from the stage. In some of these methods, the method further includes subjecting the sample to mass spectrometry analysis or sequencing analysis. In some of these methods, the tag includes a biotin derivative, a CLIP-tag, a click chemistry tag, digoxigenin tag, a HaloTag, a peptide tag, or a SNAP-tag. In some of these methods, the cleavable linker includes an azobenzene derivative, a boronic acid ester, a Dde derivative, a DNA oligomer, or a peptide. In some of these methods, the bait molecule includes an antibody, a CLIP-tag, a HaloTag, protein A, protein G, protein L, a small molecule, or a SNAP-tag. In some of these methods, the light-activated warhead includes an aryl azide, a benzophenone, or a diazirine.

Kits

Also provided herein are kits and systems for practicing the methods described herein, e.g., for generating probes, and analyzing, tagging, and labeling biomolecules. Kits can include at least one photoreactive and cleavable probe as described herein or components thereof. In some embodiments, the at least one photoreactive and cleavable probe is configured to be mildly cleavable (e.g., bioorthogonally cleavable). A kit for labeling biomolecules may include a photoreactive and cleavable probe as described herein (in a first container); and/or a connector molecule as described herein (in a second container). A kit may further include an an instructional material.

In addition, the kits will typically include an instructional material. An instructional material may include a material disclosing means for generating or modifying the one or more probes, such as e.g., attaching a bait moiety to the probe, applying the probe to a sample, conjugating the bait moiety to a prey molecule (in the sample), photocrosslinking the probe via the photoreactive warhead to a molecule of interest, photoreactively cleaving the cleavable linker via the cleavable linker bond, removing (washing away) non-photoreactive probe,

The kits may also include additional components to facilitate the particular application for which the kit is designed. Thus, for example, where a kit contains one or more photoreactive and cleavable probe for tagging and labelling biomolecules, the kit can additionally contain one or more cleavage molecule (e.g., a chemical, an endonuclease, a protease). The kit can additionally contain one or more bait molecules, such as any of those described herein (e.g., an antibody, a functional protein (e.g., protein A, protein G, a protein drug, etc.), a self-labeling protein (e.g., a CLIP-tag, a Halo-Tag, a SNAP-tag), a small molecule or drug.

The kit can additionally contain means of detecting the sample and/or detecting the label (e.g., enzyme substrates for enzymatic labels, filter sets to detect fluorescent labels, enzymes or associated detection reagents, including reagents for performing catalyzed reporter deposition (CARD) or signal amplification (e.g., avidin, Neutravidin, streptavidin, HRP, tyramide, hydrogen peroxide, etc.). The kits may additionally include wash solutions, such as blocking agents, detergents, salts (e.g., sodium chloride, potassium chloride, phosphate buffer saline (PBS)) for one or more steps (e.g., after sample fixation, after probe cleavage, etc.). A kit may include variations of wash solutions, such as concentrates of wash buffers configured to be diluted before use or components to use for making one or more wash solutions) and other reagents routinely used for the practice of a particular method. A kit may include fixatives and other sample preparation materials (e.g., ethanol, methanol, formalin, paraffin, etc.)

The kits can optionally include instructional materials teaching the use of the probes, cleavage molecules, addition of a bait molecule to a probe, and wash solution and the like.

Also provided herein are kits for preparing reagents and using probe capture by warhead-bait conjugates.

Kits for preparing reagents and using probe capture by warhead-bait conjugates can include a warhead-conjugated antibody as described above. A warhead-conjugated antibody may be a secondary antibody, though can also be other than a secondary antibody.

A kit for preparing reagents and using probe capture by warhead-bait conjugates for warhead conjugation can include one or more of (i) Multi-functional warhead molecule; (ii) Conjugation buffer stock; (iii) Stop buffer; (iv) Purification column/filter; and/or (v) Elution/storage buffer.

A kit for preparing reagents and using probe capture by warhead-bait conjugates for photoactivated tag capturing can include one or more of: (i) Photoreaction buffer stock; (ii) Biotin-tagged binding substrate (powder); (iii) Quench buffer stock; (iv) Washing buffer; (v) Post-illumination crosslinking buffer; (vi) Cleavage reagent stock; and/or (vii) Cleavage buffer stock.

A kit for preparing reagents and using probe capture for multifunctional probe for light-induced capturing as described above can include a ready-to-use multi-functional warhead-conjugated antibody.

Other kits for preparing reagents and using probe capture for multifunctional probe for light-induced capturing as described above for warhead conjugation can include one or more of: (i) Multi-functional warhead molecule; (ii) Conjugation buffer stock; (iii) Stop buffer; (iv) Purification column/filter and/or (v) Elution/storage buffer.

Other kits for preparing reagents and using probe capture for multifunctional probe for light-induced capturing as described above for photoactivated tag capturing can include one or more of: (i) Photoreaction buffer stock; (ii) Biotin-tagged binding substrate (powder); (iii) Quench buffer stock; (iv) Washing buffer(v)Post-illumination crosslinking buffer; (vi) Cleavage reagent stock; and/or (vii) Cleavage buffer stock.

### Experimental and Methods

Example 1 Demonstration of successful localized photoselective tagging of nucleolin using azo probe. FIG. 12A shows a schematic of photoselective tagging of nucleolin. Nucleolin is a protein found in the nucleolus of eukaryotic cells and involved in the synthesis of ribosomes. Azo-probe 1 was conjugated to a secondary antibody by using BCN-NHS (CAS #1516551-46-4) as additional linker between Azo-probe 1 and secondary antibody. A sample of U2OS cells was grown on a glass-bottom chamber slide and fixed with 2.4% PFA. The antibody conjugated with Azo-probe 1 was applied to the sample stained with anti-nucleolin antibody. The sample was exposed to 780 nm two-photon irradiation (200 mW, 200 μs/pixel) to photocrosslink the light-activated warhead to the antibody and subsequently incubated with 1M sodium dithionite at room temperature for over 16 h to remove non-crosslinked probes. Neutravidin conjugated to Alexa Fluor 647 dye was added and the sample assayed for the Alexa Fluor 647. Alexa Fluor 647 is a bright, far-red-fluorescent dye with excitation ideally suited for the 594 nm or 633 nm laser lines. Results are shown in the top panel of FIG. 12B. A close-up view is shown in the top right side of FIG. 12B. The characteristic nucleoli shape is observed. A side view is shown in the bottom right of FIG. 12B. The bottom of FIG. 12B shows a control region treated the same as in the top panel except that the sample shown in the bottom panel was not exposed to photoactivating light. No significant staining was observed.

### Example 2

Preparation of BCN-antibody:

1. For 100 μl of reaction, prepare 70 μl antibody (1.2-1.5 μg/μl) of solution. 2. Add 10 μl of 1M sodium bicarbonate (or 1M borate buffer, final 50-100 mM) and BCN-NHS (Sigma-Aldrich #744867, final concentration: 200 μM). Adjust the final volume to 100 μl with ddH2O. 3. Mix gently by inverting the tube a few times and mildly spin down. 4. Incubate on shaker/mixer for 1 hour at room temperature. Avoid from light if needed. 5. Stop the reaction by adding 10 μl of 1M glycine and react for another 30-60 minutes at room temperature. 6. Remove non-conjugated small molecules by resin filtration using desalting column.

Preparation of Probe 3-antibody conjugate: 7. Mix 0.5-1 μg/μl antibody with probe 3 (final concentration: 100 μM), react overnight at 4° C. 8. Remove non-conjugated small molecules by resin filtration using desalting column.

Photoselective labeling: 9. Treat nucleolin-stained cells with Probe 3-antibody and DRAQ5 (nucleus marker) in PBS solution supplement with 0.1% triton for 60 min. 10. Wash the sample with PBS solution supplement with 0.1% triton and fix the sample with 2.4% PFA. 11. Define desired area and label the Probe 3-antibody stained nucleolin within the selected area with 160-200 mW pulsed laser at 780 nm. 12. Wash the labeled samples with PBS and incubate with 1M sodium dithionite overnight at RT. 13. Check the labeling by staining with NeutrAvidin-Dy550 conjugates (1:200).

### Example 3

Preparation of Probe IV N-TEV

Pre-conjugated peptides N-TEV were dissolved in DMSO/Water (1/1) to 1 mM. N-Succinimidyl 4-Benzoylbenzoate (TCI #S0863) were dissolved in pure anhydrous DMSO to 2 mM. 10 μL of N-TEV stock solution were mixed with 10 μL of N-Succinimidyl 4-Benzoylbenzoate stock solution, 10 μL of 1M sodium borate buffer (pH=8.5), 70 μL of DMSO/Water (1/1) and react for 2 h at room temperature. The reaction was quenched by adding 10 μL of 1M glycine solution and validated with MALDI-MS.

### Example 4

### Experiment Procedure for Preparing and Using Probe Capture by Warhead-Bait Conjugates

Conjugation of Photoreactive Warheads with Secondary Antibody

Mix materials and reagents as in Table 2:

| Stock | Concentration | | Volume (μL) |
|---|---|---|---|
| Unconjugated antibody (Ab) | 1.3 | μg/μL | 77 |
| NHS-Tris-Azide (CP-2214, Conju-Probe) | 100 | mM | 0.83 |

| | | |
|---|---|---|
| Borate buffer | 1M | 10 |
| PBS | 1× | 12.2 |

Mix (100 rpm) the mixture at room temperature (RT) for 1 h.

Add 400 μL of 100 mM glycine to quench the reaction.

Purify the Ab-Tris-Azide conjugate with PD Minitrap™ G-25 column.

Concentrate the eluate to 100 μL in volume using 10 kDa Amicon Ultra-0.5 centrifugal filter.

Add Hydroxyl di-benzophenone (PEG)₃ alkyne (900610, Sigma) into 100 μL of concentrated Ab-Tris-Azide to a final concentration of 0.5 mM.

Add CuSO₄ to a final concentration of 2 mM, THPTA to a final concentration of 10 mM and sodium ascorbate to a final concentration of 100 mM, respectively.

Mix via vortex (100 rpm) and incubate the solution for 4 h at RT, followed by another 14 h (overnight) incubation at 4° C.

Purify the Ab-Tris-Azide-BzP with PD Minitrap G-25 column.

Sample Preparation

Cells on a chambered coverslip with 4 wells (80427, ibidi) were fixed with 2.4% paraformaldehyde/PBS for 10 min at RT and permeabilized through incubation with 0.5% Triton X-100 in PBS for 10 min at RT.

Cells were then blocked with 3% BSA/0.1% PBST for 1 h, then treated with 0.002% streptavidin in 0.1% PBST for 30 min at RT to block the endogenous biotin. After three times of 0.1% PBST washes, 40 μM biotin was added into the wells for blocking residual streptavidin.

Immunochemistry

Cells were stained with proper concentration (normally 1-10 ug/mL) of primary antibody of desired target in blocking buffer containing 3% BSA and 0.1% PBST for 1 h at RT, or overnight at 4° C.

Warhead-conjugated secondary antibody (Ab-Tris-Azide-diBzP in this embodiment) was prepared to 10 μg/mL in 0.1% PBST and was incubated with the sample for 1 hr at room temperature, or at 4° C. overnight for binding primary antibody.

Light-driven tag capturing

Another secondary antibody with fluorophore was used to visualize target protein (if needed) for choosing the region of interest (ROI). On the other hand, a biotin-tagged/warhead binding peptide molecule with sequence “Biotin-FMGMGGGGS-Cysteic acid-F” (SEQ ID NO: 16), was prepared to 500 μM with 0.1% PBST and was added into the chambered coverslip.

The peptide molecule serves as a substrate/binding target of the warheads on the antibody and as a biotin-tagged probe to bind to the areas through light illumination (one-photon or multiphoton).

After illumination, the labeled cells were washed three times with 0.1% PBST (15 min/each) followed by an overnight washing.

Validation of light-driven tag capturing

Neutravidin-fluorophore conjugate (1 ug/mL in 3% BSA/PBST, 1 h) was used to probe the biotin-labeled region after illumination. Results are shown in FIG. 17D.

### Example 5

Multifunctional probe for light-induced capturing

Conjugation of multi-functional warhead with antibody

100 mM Sulfo-SMCC: dissolve 5 mg of Sulfo-SMCC in 115 μL DMSO.

10 mM Sulfo-SMCC: mix 1 μL of 100 mM Sulfo-SMCC and 9 μL of PBS.

10 mM multi-functional peptide: dissolve 4 mg of peptide in 180 μL DMSO.

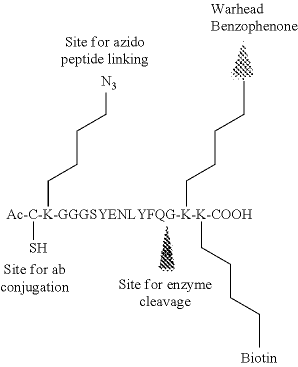

Multi-functional peptide (SEQ ID NO: 41)

Mix 5 μL of 10 mM Sulfo-SMCC and 5 μL of 10 mM multi-functional peptide-based warhead, and let the solution stands for 10 min at RT.

Add 100 μg of secondary antibody(ab) into the tube of last step, and make the final volume as 100 μL with PBS.

Vortex (100 rpm) the mixture at RT for 1 h in the dark.

| Stock | Concentration | | Volume (μL) |
|---|---|---|---|
| Secondary ab | 1.8 | μg/μL | 55 |
| SMCC (22360, Thermo Scientific) | 10 | mM | 5 |
| Multi-functional peptide | 10 | mM | 5 |

| | | |
|---|---|---|
| PBS | 1× | 35 |

Purify the antibody-peptide conjugates using PD Miditrap G-25 column.

(Procedures for sample preparation, immunochemistry and light-driven tag capturing are as described above in the section “Experiment Procedure for preparing and using probe capture by warhead-bait conjugates”

Post-illumination crosslinking

After the light illumination, cells were washed three times with 0.1% PBST (15 min/wash).

Add crosslinking reagent mix into the chambered coverslip, and incubate it for 1 h at RT followed by 16 h at 4° C. on the seesaw shaker (15 rpm).

(Crosslinking regent mix: 1 mM Bis-propargyl-PEG12, 2 mM CuSO₄, 10 mM THPTA, 100 mM Sodium ascorbate)

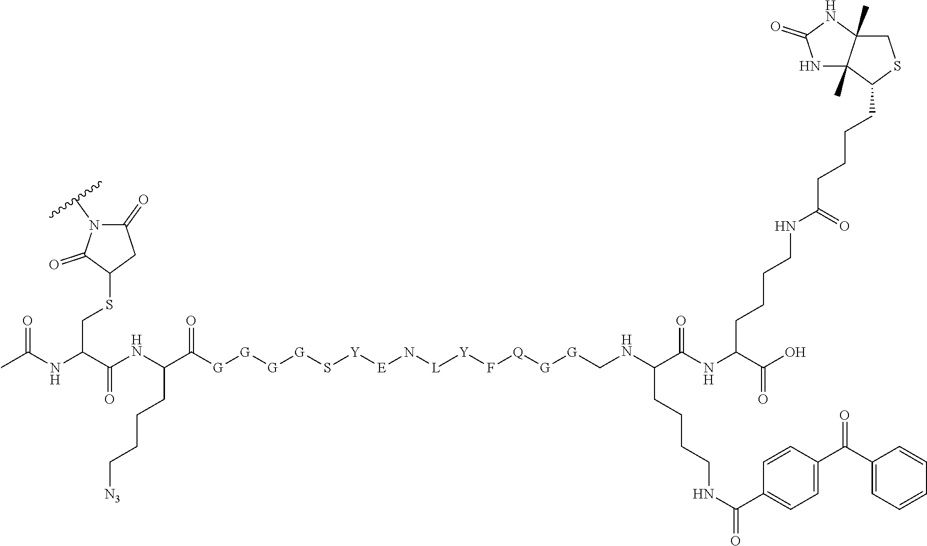

Crosslink azido peptide and multi-functional peptide (SEQ ID NO: 37)

TEV digestion

Add digestion buffer into the chambered coverslip, and incubate it for 16 h at 30° C. on the seesaw shaker (15 rpm). After the digestion, wash the cells three times with 0.1% PB ST (15 min/each).

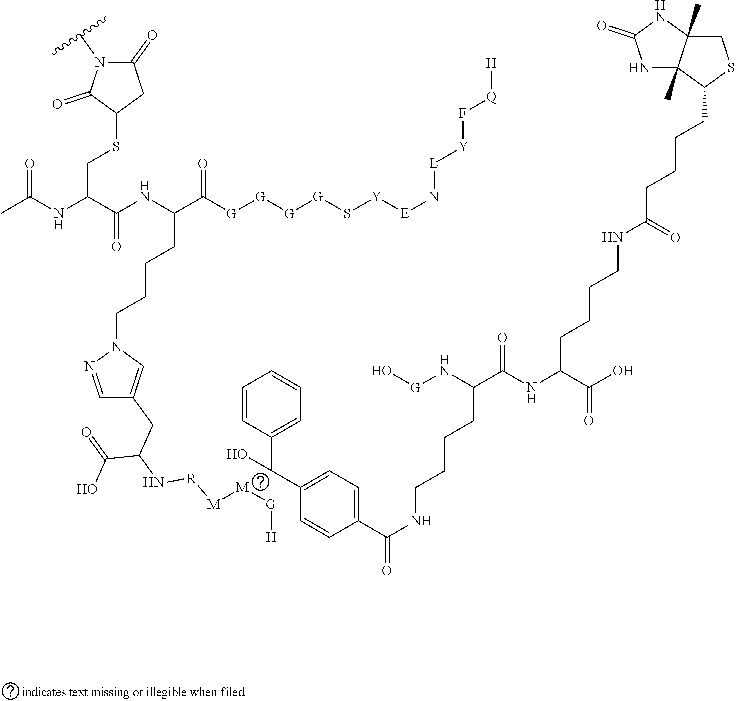

TEV digestion (SEQ ID NOS 39-40, respectively, in order of appearance)

(Digestion buffer: 300 units/mL of TEV in 1× digestion buffer)

(Procedures for validation are as described above in the section “Experiment Procedure for preparing and using probe capture by warhead-bait conjugates”

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

In general, any of the apparatuses and methods described herein should be understood to be inclusive, but all or a sub-set of the components and/or steps may alternatively be exclusive, and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

1. A photoreactive and cleavable probe of formula (I): 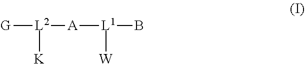
wherein
a cleavable linker of the probe comprises:
an L¹ portion at a proximal region of the cleavable linker;
an L² portion at a distal region of the cleavable linker;
an A portion comprising a cleavage site;
W includes a light-activated warhead covalently bound to the proximal region of the cleavable linker;
B includes a tag bound to the proximal region of the cleavable linker;
K includes a crosslinkable group bound to the distal region of the cleavable linker; and
G comprises a bait molecule bound to the distal region of the cleavable linker.

2. The photoreactive and cleavable probe of claim 1, wherein the bait molecule comprises an antibody and the antibody is bound to the distal region of the cleavable linker.

3. The photoreactive and cleavable probe of claim 1, wherein the bait molecule comprises a secondary antibody and the secondary antibody is bound to the distal region of the cleavable linker.

4. The photoreactive and cleavable probe of claim 1, wherein the bait molecule comprises a CLIP-tag, a HaloTag, protein A, protein G, protein L, an RNA molecule, a small molecule, or a SNAP-tag.

5. The photoreactive and cleavable probe of claim 1, wherein the crosslinkable group comprises a click chemistry-based moiety.

6. The photoreactive and cleavable probe of claim 1, wherein the crosslinkable group comprises a bioorthogonal moiety.

7. The photoreactive and cleavable probe of claim 1, wherein the crosslinkable group comprises a strained alkyne, a terminal alkyne, an azide, a tetrazine, a strained alkene, or a 2-cyano-6-aminobenzothiazole (CBT) moiety.

8. The photoreactive and cleavable probe of claim 1, wherein the cleavable linker comprises a cleavable linker bond other than a disulfide bond.

9. The photoreactive and cleavable probe of claim 1, wherein the cleavable linker comprises an azobenzene derivative, a boronic acid ester, a Dde derivative, a DNA oligomer, or a specifically cleavable peptide.

10.-11. (canceled)

12. The photoreactive and cleavable probe of claim 1, wherein the cleavable linker comprises a specifically cleavable peptide.

13. The photoreactive and cleavable probe of claim 12, wherein the cleavable linker comprises a bioorthogonal protease-cleavable peptide chain.

14. The photoreactive and cleavable probe of claim 1, wherein the cleavable linker comprises a human rhinovirus 3C (HRV 3C) protease recognition sequence or a tobacco etch virus (TEV) protease recognition sequence.

15. The photoreactive and cleavable probe of claim 1, wherein the tag comprises a biotin derivative, a click chemistry tag, a CLIP-tag, a digoxigenin tag, a HaloTag, a peptide tag, or a SNAP-tag.

16. The photoreactive and cleavable probe of claim 1, wherein the tag comprises a click chemistry tag, and the click chemistry tag comprises an alkyne-based or azide-based moiety.

17. The photoreactive and cleavable probe of claim 1, wherein the tag comprises a click chemistry tag, and the click chemistry tag includes the moiety of

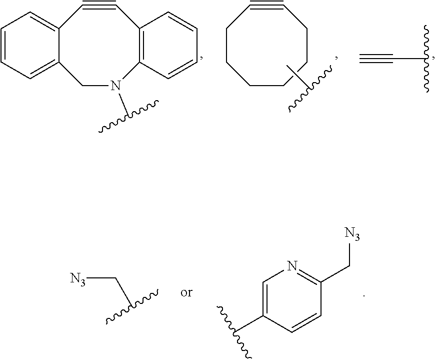

18. The photoreactive and cleavable probe of claim 1, wherein the tag comprises a biotin derivative, and the biotin derivative includes the moiety of

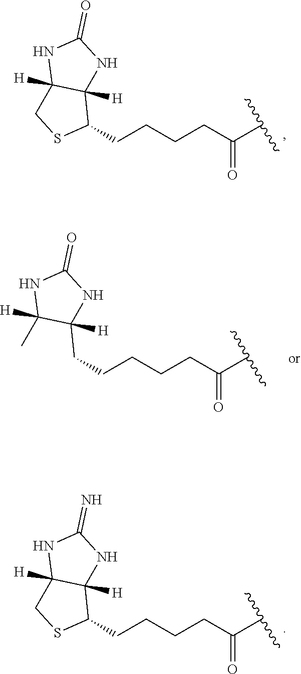

19. The photoreactive and cleavable probe of claim 1, wherein the light-activated warhead comprises an aryl azide, a benzophenone, or a diazirine.

20.-25. (canceled)

26. The photoreactive and cleavable probe of claim 1, wherein the L1 portion includes a moiety of formula (I-1), and B and W are bound to the proximal region of the cleavable linker through the moiety of formula (I-1): 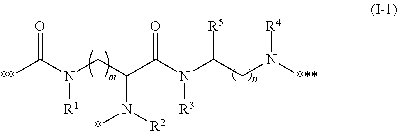
wherein m and n each independently are 1, 2, 3, 4, 5, or 6;
the symbol * is a first attachment site for a proximal end of the A portion;
the symbol ** is a second attachment site for one of either the tag or the light-activated warhead;
the symbol *** is a third attachment site for the other one of the tag or the light-activated warhead;
R¹, R², R³ and R⁴ each independently are hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, or a nitrogen protecting group; and
R⁵ is —COORa, wherein Ra is hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, or an oxygen protecting group.

27. The photoreactive and cleavable probe of claim 1, wherein the L² portion includes a moiety of formula (I-2), and the G and the K are bound to the distal region of the cleavable linker through the moiety of formula (I-2): 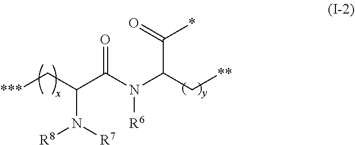
wherein x and y each independently are 1, 2, 3, 4, 5, or 6;
the symbol * is a first attachment site for a distal end of the A portion;
the symbol ** is a second attachment site for one of the crosslinkable group and the bait molecule;
the symbol *** is a third attachment site for another one of the crosslinkable group and the bait molecule; and
R⁶, R⁷ and R⁸ each independently are hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, or a nitrogen protecting group.

28. The photoreactive and cleavable probe of claim 1, which comprises the following structure (SEQ ID NO: 37):

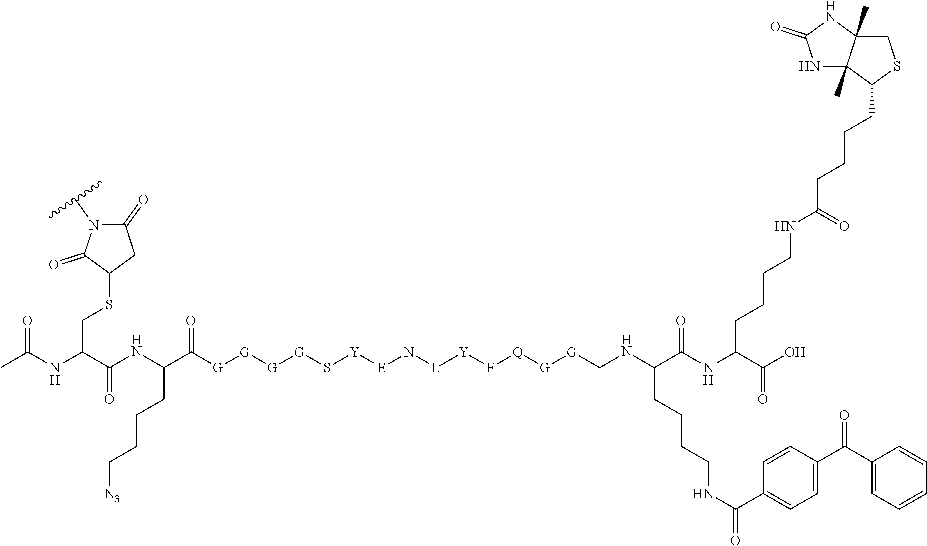

29.-81. (canceled)


SEQUENCE LISTING (43 sequences)

SEQ ID NO: 1 - protein, 6 aa, synthetic construct
HHHHHH

SEQ ID NO: 2 - protein, 5 aa, synthetic construct
DDDDK

SEQ ID NO: 3 - protein, 6 aa, synthetic construct
LVPRGS

SEQ ID NO: 4 - protein, 8 aa, synthetic construct
LEVLFQGP

SEQ ID NO: 5 - protein, 7 aa, synthetic construct
ENLYFQS

SEQ ID NO: 6 - protein, 7 aa, synthetic construct
ENLYFQG

SEQ ID NO: 7 - protein, 6 aa, synthetic construct
LVPRGS

SEQ ID NO: 8 - protein, 13 aa, synthetic construct
GRRRYLEVLFQGP

SEQ ID NO: 9 - protein, 13 aa, synthetic construct
LEVLFQGPYRRRG

SEQ ID NO: 10 - protein, 11 aa, synthetic construct
ENLYFQGGGGS

SEQ ID NO: 11 - protein, 11 aa, synthetic construct
LVPRGSYRRRG

SEQ ID NO: 12 - protein, 6 aa, synthetic construct
LVPRGS

SEQ ID NO: 13 - protein, 13 aa, synthetic construct
LEVLFQGPGGGGS

SEQ ID NO: 14 - protein, 13 aa, synthetic construct
ENLYFQGGYRRRG

SEQ ID NO: 15 - protein, 13 aa, synthetic construct
GGGGSYENLYFQG

SEQ ID NO: 16 - protein, 11 aa, synthetic construct
  SITE 1: note = Biotin phenylalanine
  SITE 10: note = Cysteic acid
FMGMGGGGSXF

SEQ ID NO: 17 - protein, 11 aa, synthetic construct
  SITE 1: note = Biotin glycine
  SITE 10: note = Cysteic acid
GFMMGGGGSXF

SEQ ID NO: 18 - protein, 12 aa, synthetic construct
  SITE 1: note = Biotin lysine
  SITE 11: note = Cysteic acid
KPKDTLMISRXF

SEQ ID NO: 19 - protein, 11 aa, synthetic construct
  SITE 1: note = Desthiobiotin phenylalanine
  SITE 10: note = Cysteic acid
FMGMGGGGSXF

SEQ ID NO: 20 - protein, 5 aa, synthetic construct
  SITE 1: note = Biotin glycine
GMGGE

SEQ ID NO: 21 - protein, 5 aa, synthetic construct
  SITE 1: note = Biotin methionine
MGMGE

SEQ ID NO: 22 - protein, 40 aa, synthetic construct
  VARIANT 1..40: note = This sequence may encompass 1-8 GGGGS repeating units
GGGGSGGGGSGGGGSGGGGSGGGGSGGGGSGGGGSGGGGS

SEQ ID NO: 23 - protein, 5 aa, synthetic construct
GGGGS

SEQ ID NO: 24 - protein, 5 aa, synthetic construct
  SITE 5: note = Propargylglycine
GMMRX

SEQ ID NO: 25 - protein, 5 aa, synthetic construct
  SITE 1: note = Acetyl glycine
  SITE 5: note = Lysine polyethylene glycol alkyne
GMMRK

SEQ ID NO: 26 - protein, 5 aa, synthetic construct
  SITE 1: note = Acetyl glycine
  SITE 5: note = Azidolysine
GMMRX

SEQ ID NO: 27 - protein, 8 aa, synthetic construct
  SITE 1: note = Acetyl aspartic acid
  SITE 8: note = Azidolysine
DTLMISRX

SEQ ID NO: 28 - protein, 15 aa, synthetic construct
  SITE 1: note = Acetyl azidoalanine
  SITE 15: note = Lysine 1,2-ethylene diamine biotin
XGRRRYLEVLFQGPK

SEQ ID NO: 29 - protein, 14 aa, synthetic construct
  SITE 1: note = Lysine linked to biotin through amide functionality
  SITE 14: note = Azidolysine amide
KLEVFQGPYRRRGX

SEQ ID NO: 30 - protein, 13 aa, synthetic construct
  SITE 1: note = Lysine linked to biotin through amide functionality
  SITE 13: note = Azidolysine amide
KENLYFQGGGGSX

SEQ ID NO: 31 - protein, 13 aa, synthetic construct
  SITE 1: note = Lysine linked to biotin through amide functionality
  SITE 13: note = Azidolysine amide
KLVPRGSYRRRGX

SEQ ID NO: 32 - protein, 8 aa, synthetic construct
  SITE 1: note = Lysine linked to biotin through amide functionality
  SITE 8: note = Azidolysine amide
KLVPRGSX

SEQ ID NO: 33 - protein, 15 aa, synthetic construct
  SITE 1: note = Lysine linked to biotin through amide functionality
  SITE 15: note = Propargylglycine amide
KLEVLFQGPGGGGSX

SEQ ID NO: 34 - protein, 15 aa, synthetic construct
  SITE 1: note = Lysine linked to biotin through amide functionality
  SITE 15: note = Propargylglycine amide
KENLYFQGGYRRRGX

SEQ ID NO: 35 - protein, 15 aa, synthetic construct
  SITE 1: note = Acetyl azidoalanine
  SITE 15: note = Lysine 1,2-ethylene diamine biotin
XGGGGSYENLYFQGK

SEQ ID NO: 36 - protein, 13 aa, synthetic construct
  SITE 1: note = Lysine linked to biotin through amide functionality with 6-N-acetyl benzophenone substitution
  SITE 13: note = Azidolysine amide
KENLYFQGGGGSX

SEQ ID NO: 37 - protein, 17 aa, synthetic construct
  SITE 1: note = Acetyl N-hydroxy succinimide activated cysteine
  SITE 2: note = Azidolysine
  SITE 16: note = Lysine 6-N-acetyl benzophenone
  SITE 17: note = Lysine linked to biotin through amide functionality
CXGGGGSYENLYFQGKK

SEQ ID NO: 38 - protein, 6 aa, synthetic construct
  SITE 6: note = Propargylglycine
HGMMRX

SEQ ID NO: 39 - protein, 15 aa, synthetic construct
  SITE 1: note = Acetyl N-hydroxy succinimide activated cysteine
  SITE 2: note = Lysine N-substituted-1,2,3-triazole
CKGGGGSYENLYFQH

SEQ ID NO: 40 - protein, 6 aa, synthetic construct
  VARIANT 3: note = Methionine with 4-(benzophenone methylthio) substitution or (4 benzophenone, 4 methylthio) substitution
  SITE 6: note = Alanine 4-substituted-1,2,3-triazole
HGMMRA

SEQ ID NO: 41 - protein, 16 aa, synthetic construct
  SITE 1: note = Acetyl cysteine
  SITE 2: note = Azidolysine
  SITE 15: note = Lysine 6-N-acetyl benzophenone
  SITE 16: note = Lysine linked to biotin through amide functionality
CXGGGSYENLYFQGKK

SEQ ID NO: 42 - protein, 5 aa, synthetic construct
HHHHH

SEQ ID NO: 43 - protein, 10 aa, synthetic construct
  VARIANT 1..10: note = This sequence may encompass 7-10 residues
HHHHHHHHHH